(19) United States
(12) Patent Application Publication
(10) Pub. No.: US 2021/0187111 A1
(43) Pub. Date: Jun. 24, 2021

(54) COMPOSITIONS AND METHODS FOR DISRUPTING A MACROPHAGE NETWORK

(71) Applicant: Scripps Health, San Diego, CA (US)

(72) Inventors: Faith BARNETT, San Diego, CA (US); Mauricio ROSENFELD, San Diego, CA (US)

(21) Appl. No.: 17/150,703

(22) Filed: Jan. 15, 2021

Related U.S. Application Data
(63) Continuation of application No. PCT/US2019/041824, filed on Jul. 15, 2019.
(60) Provisional application No. 62/699,427, filed on Jul. 17, 2018.

(51) Int. Cl. A61K 47/54; A61K 45/06; A61P 35/00; A61K 47/64; A61K 47/61

(52) U.S. Cl. CPC A61K 47/549; A61K 45/06; A61K 47/61; A61K 47/6435; A61P 35/00

(57) ABSTRACT

Described herein are methods and compositions for disrupting a macrophage network. Described herein are targeting agents capable of targeting one or more cells of a macrophage network. Methods for treating various diseases, such as cancer and granulomatous diseases are provided.

### CROSS-REFERENCE

This application is a continuation of International Application No. PCT/US2019/041824, filed on Jul. 15, 2019, which claims the benefit of U.S. Provisional Application No. 62/699,427, filed on Jul. 17, 2018, the contents of each are incorporated herein by reference in their entirety.

### SUMMARY OF THE INVENTION

In some aspects, provided herein, is a method of treating a granulomatous disease in a subject comprising disrupting a macrophage network comprising one or more macrophages. In some embodiments, the granulomatous disease is an infectious disease. In some embodiments, the infectious disease is tuberculosis, histoplasmosis, cryptococcis, coccidiomycosis, leprosy, blastomycoccis, or cat scratch disease. In some embodiments, the granulomatous disease is sarcoidosis, berylliosis, granulomatosis with polyangiitis, giant cell tumor disease, Rosai-Dorfman disease, rheumatoid arthritis, or Crohn's disease. In some embodiments, the disrupting comprises administering a therapeutically effective amount of a compound to the subject. In some embodiments, the compound is capable of killing the one or more macrophages. In some embodiments, the compound comprises a targeting moiety that targets the one or more macrophages.

In some aspects, provided herein, is a method of treating cancer in a subject comprising disrupting a macrophage network comprising one or more macrophages. In some embodiments, the disrupting comprises administering a therapeutically effective amount of a compound to the subject. In some embodiments, the compound is capable of killing the one or more macrophages. In some embodiments, the compound comprises a targeting moiety that targets the one or more macrophages.

In some embodiments of the above aspects, the targeting moiety is a CD206 ligand. In some embodiments, the CD206 ligand comprises at least a portion of mannose, galactose, collagen, fucose, sulfated N-acetylgalactosamine, N-acetylglucosamine, luteinizing hormone, thyroid stimulating hormone, or a chondroitin sulfate. In some embodiments, the targeting moiety is a CD204 ligand. In some embodiments, the CD204 ligand comprises at least a portion of lipid A, oxidized low-density lipoprotein (LDL), acetylated LDL, malondialdehyde modified LDL, maleylated LDL, lysophasphatidylcholine, phophatidic acid, cholesterol, Apo A-I, Apo E, glycated type IV collagen, modified collagen type I, III and IV, biglycan, decorin, albumin, advanced glycation end product bovine serum albumin, b-amyloid fibrils, calreticulin, gp96, an HSP70 protein, a lipopolysaccharide, lymphotoxin-alpha, CpG DNA, calciprotein particles, a Neisseria meningitides surface protein, C reactive protein, hepatitis C virus NS3 protein, or Tamm-Horsfall protein. In some embodiments, the targeting moiety is a CD163 ligand. In some embodiments, the CD163 ligand comprises at least a portion of haptoglobin, hemoglobin, tumor necrosis factor-α (TNF-α)-like weak inducer of the apoptosis (TWEAK), gram positive and gram negative bacteria, or casein kinase II subunit beta. In some embodiments, the compound further comprises a cytotoxic agent or a macrophage polarizing agent. In some embodiments, the cytotoxic agent is a chemotherapeutic agent, an anti-tubulin agent, a DNA modifying agent, or a small interfering ribonucleic acid. In some embodiments, the cytotoxic agent is selected from the group consisting of an auristatin, a dolastatin, auristatin E, Monomethyl auristatin E (MMAE), Monomethyl auristatin F (MMAF), dimethylvaline-valine-dolaisoleuine-dolaproine-phenylalanine-p-phenylenediamine (AFP), 5-benzoylvaleric acid-auristatin E ester (AEVB), auristatin EB (AEB), Ansamitocin, Mertansine/emtansine (DM1), ravtansine/soravtansine (DM4), Duocamycin, Calicheamicin, and pyrrolobenzodiazepines. In some embodiments, the cytotoxic agent is MMAE. In some embodiments, the macrophage polarizing agent is CpG DNA. In some embodiments, the compound comprises a structure of T-B-P, where T is the targeting moiety, B is a backbone, and P is the cytotoxic agent or the macrophage polarizing agent. In some embodiments, the cytotoxic agent or the macrophage polarizing agent is attached to the backbone via a cleavable linker L. In some embodiments, the cleavable linker is capable of being cleaved by a protease. In some embodiments, the protease is a lysosomal protease or an endosomal protease. In some embodiments, the cleavable linker is capable of being cleaved by a pH change. In some embodiments, the cleavable linker comprises a disulfide bond. In some embodiments, the backbone is a peptide backbone. In some embodiments, the backbone is a dextran backbone. In some embodiments, the compound is less than about 20 kDa, less than about 15 kDa, less than about 10 kDa, less than about 5 kDa, less than about 4 kDa, less than about 3 kDa, less than about 2 kDa, less than about 1 kDa, or less than about 0.5 kDa in size.

In some aspects, provided herein, is a method of delivering an agent to a tumor comprising administering a compound comprising the agent and a targeting moiety to a subject, wherein the compound is capable of entering and perfusing through a macrophage network. In some embodiments, the agent is a cytotoxic agent. In some embodiments, the cytotoxic agent is a chemotherapeutic agent, an anti-tubulin agent, a DNA modifying agent, or a small interfering ribonucleic acid. In some embodiments, the cytotoxic agent is selected from the group consisting of an auristatin, a dolastatin, auristatin E, Monomethyl auristatin E (MMAE), Monomethyl auristatin F (MMAF), dimethylvaline-valine-dolaisoleuine-dolaproine-phenylalanine-p-phenylenediamine (AFP), 5-benzoylvaleric acid-auristatin E ester (AEVB), auristatin EB (AEB), Ansamitocin, Mertansine/emtansine (DM1), ravtansine/soravtansine (DM4), Duocamycin, Calicheamicin, and pyrrolobenzodiazepines. In some embodiments, the agent is an imaging agent. In some embodiments, the imaging agent is 5-carboxyfluorescein, fluorescein-5-isothiocyanate, fluorescein-6-isothiocyanate, 6-carboxyfluorescein, tetramethylrhodamine-6-isothiocyanate, 5-carboxytetramethylrhodamine, 5-carboxy rhodol derivatives, tetramethyl and tetraethyl rhodamine, diphenyldimethyl and diphenyldiethyl rhodamine, dinaphthyl rhodamine, rhodamine 101 sulfonyl chloride, Cy3, Cy3B, Cy3.5, Cy5, Cy5.5, Cy7, DyLight650, IRDye650, IRDye680, DyLight750, Alexa Fluor 647, Alexa Fluor 750, IR800CW, ICG, Green Fluorescent Protein, EBFP, EBFP2, Azurite, mKalamal, ECFP, Cerulean, CyPet, YFP, Citrine, Venus, YPet, a gadolinium chelate, an iron oxide particle, a super paramagnetic iron oxide particle, an ultra-small paramagnetic particle, a manganese chelate, gallium containing agent, 64Cu diacetyl-bis(N4-methylthiosemicarbazone), 18F-fluorodeoxyglucose, 18F-fluoride, 3′-deoxy-3′-[18F]fluorothymidine, 18F-fluoromisonidazole, technetium-99m, thallium, iodine, barium-sulphate, or a combination thereof. In some embodiments, the macrophage network comprises tumor associated macrophages. In some embodiments, the compound is capable of targeting the tumor associated macrophages. In some embodiments, the compound is less than about 20 kDa, less than about 15 kDa, less than about 10 kDa, less than about 5 kDa, less than about 4 kDa, less than about 3 kDa, less than about 2 kDa, less than about 1 kDa, or less than about 0.5 kDa in size. In some embodiments, compound comprises a structure of T-B-A, where T is the targeting moiety, B is a backbone, and A is the agent. In some embodiments, the backbone is a peptide backbone. In some embodiments, the backbone is a dextran backbone. In some embodiments, the agent is attached to the backbone via a linker. In some embodiments, the linker is a cleavable linker. In some embodiments, the cleavable linker is capable of being cleaved by a protease. In some embodiments, the protease is a lysosomal protease or an endosomal protease. In some embodiments, the cleavable linker is capable of being cleaved by a pH change. In some embodiments, the cleavable linker comprises a disulfide bond. In some embodiments, the targeting moiety is a CD206 ligand. In some embodiments, the CD206 ligand comprises at least a portion of mannose, galactose, collagen, fucose, sulfated N-acetylgalactosamine, N-acetylglucosamine, luteinizing hormone, thyroid stimulating hormone, or a chondroitin sulfate. In some embodiments, the targeting moiety is a CD204 ligand. In some embodiments, the CD204 ligand comprises at least a portion of lipid A, oxidized low-density lipoprotein (LDL), acetylated LDL, malondialdehyde modified LDL, maleylated LDL, lysophasphatidylcholine, phophatidic acid, cholesterol, Apo A-I, Apo E, glycated type IV collagen, modified collagen type I, III and IV, biglycan, decorin, albumin, advanced glycation end product bovine serum albumin, b-amyloid fibrils, calreticulin, gp96, an HSP70 protein, a lipopolysaccharide, lymphotoxin-alpha, CpG DNA, calciprotein particles, a Neisseria meningitides surface protein, C reactive protein, hepatitis C virus NS3 protein, or Tamm-Horsfall protein. In some embodiments, the targeting moiety is a CD163 ligand. In some embodiments, the CD163 ligand comprises at least a portion of haptoglobin, hemoglobin, tumor necrosis factor-α (TNF-α)-like weak inducer of the apoptosis (TWEAK), gram positive and gram negative bacteria, or casein kinase II subunit beta.

In some aspects, provided herein, is a method of treating a granulomatous disease in a subject comprising: (a) disrupting a macrophage network comprising one or more macrophages; (b) and administering a therapeutically effective amount of a therapeutic agent. In some embodiments, the therapeutic agent is an antibacterial agent, an anti-inflammatory agent, or a combination thereof. In some embodiments, the antibacterial agent comprises an antibiotic. In some embodiments, the antibacterial agent comprises isoniazid, rifampin, ethambutol, purazinamide, amikacin, kanamycin, capreomycin, or combinations thereof. In some embodiments, the therapeutic agent is an anti-inflammatory agent. In some embodiments, the anti-inflammatory agent comprises a nonsteroidal anti-inflammatory drug (NSAID), a glucocorticoid, or a disease-modifying agent of rheumatoid diseases (DMARD). In some embodiments, the NSAID is asprin, celecoxib, diclofenac, diflunisal, etodolac, ibuprofen, indomethacin, ketoprofin, ketorolac, nabumetone, naproxen, oxaprozin, piroxicam, salsalate, sulindac, or tolmetin. In some embodiments, the glucocorticoid is beclomethasone, betamethasone, budesonide, cortisone, dexamethasone, hydrocortisone, methylprednisolone, prednisolone, prednisone, or triamcinolone. In some embodiments, the DMARD is methotrexate, sulfasalazine, hydroxychloroquinine, leflunomide, azathioprine, cyclosporine, etanercept, adalimumab, infliximab, certolizumab pegol, or golimumab.

In some aspects, provided herein, is a method of treating cancer in a subject comprising: (a) disrupting a macrophage network comprising one or more tumor associated macrophages; and (b) administering a therapeutically effective amount of an anti-cancer therapy. In some embodiments, the anti-cancer therapy is surgery, radiation therapy, immunotherapy, chemotherapy, targeted therapy, or hormone therapy. In some embodiments, the immunotherapy comprises treatment with an antibody, an antibody-drug conjugate, an antibody-like molecule, an antibody fragment, a recombinant protein, a T-cell receptor, a T-cell, a cancer vaccine, a cytokine, or Bacillus Calmette-Guérin (BCG). In some embodiments, the T-cell is a chimeric antigen receptor (CAR) T-cell. In some embodiments, the chemotherapy comprises treatment with a chemotherapeutic agent. In some embodiments, the hormone therapy comprises treatment with tamoxifen, toremifene, fulvestrant, letrozole, anastrozole, exemestane, leuprolide, goserelin, triptorelin, or histrelin. In some embodiments, the disrupting comprises administering a therapeutically effective amount of a compound to the subject. In some embodiments, the compound is capable of killing the one or more macrophages. In some embodiments, the compound comprises a targeting moiety that targets the one or more macrophages. In some embodiments, the targeting moiety is a CD206 ligand. In some embodiments, the CD206 ligand comprises at least a portion of mannose, galactose, collagen, fucose, sulfated N-acetylgalactosamine, N-acetylglucosamine, luteinizing hormone, thyroid stimulating hormone, or a chondroitin sulfate. In some embodiments, the targeting moiety is a CD204 ligand. In some embodiments, the CD204 ligand comprises at least a portion of lipid A, oxidized low-density lipoprotein (LDL), acetylated LDL, malondialdehyde modified LDL, maleylated LDL, lysophasphatidylcholine, phophatidic acid, cholesterol, Apo A-I, Apo E, glycated type IV collagen, modified collagen type I, III and IV, biglycan, decorin, albumin, advanced glycation end product bovine serum albumin, b-amyloid fibrils, calreticulin, gp96, an HSP70 protein, a lipopolysaccharide, lymphotoxin-alpha, CpG DNA, calciprotein particles, a Neisseria meningitides surface protein, C reactive protein, hepatitis C virus NS3 protein, or Tamm-Horsfall protein. In some embodiments, the targeting moiety is a CD163 ligand. In some embodiments, the CD163 ligand comprises at least a portion of haptoglobin, hemoglobin, tumor necrosis factor-α (TNF-α)-like weak inducer of the apoptosis (TWEAK), gram positive and gram negative bacteria, or casein kinase II subunit beta. In some embodiments, the compound comprises a cytotoxic agent. In some embodiments, the cytotoxic agent is a chemotherapeutic agent, an anti-tubulin agent, a DNA modifying agent, or a small interfering ribonucleic acid. In some embodiments, the cytotoxic agent is selected from the group consisting of an auristatin, a dolastatin, auristatin E, Monomethyl auristatin E (MMAE), Monomethyl auristatin F (MMAF), dimethylvaline-valine-dolaisoleuine-dolaproine-phenylalanine-p-phenylenediamine (AFP), 5-benzoylvaleric acid-auristatin E ester (AEVB), auristatin EB (AEB), Ansamitocin, Mertansine/emtansine (DM1), ravtansine/soravtansine (DM4), Duocamycin, Calicheamicin, and pyrrolobenzodiazepines. In some embodiments, the compound comprises a structure of T-B-P, where T is the targeting moiety, B is a backbone, and P is the cytotoxic agent. In some embodiments, the cytotoxic agent is attached to the backbone via a cleavable linker L. In some embodiments, the cleavable linker is capable of being cleaved by a protease. In some embodiments, the protease is a lysosomal protease or an endosomal protease. In some embodiments, the cleavable linker is capable of being cleaved by a pH change. In some embodiments, the cleavable linker comprises a disulfide bond. In some embodiments, the backbone is a peptide backbone. In some embodiments, the backbone is a dextran backbone. In some embodiments, the compound comprises an imaging agent. In some embodiments, the imaging agent is 5-carboxyfluorescein, fluorescein-5-isothiocyanate, fluorescein-6-isothiocyanate, 6-carboxyfluorescein, tetramethylrhodamine-6-isothiocyanate, 5-carboxytetramethylrhodamine, 5-carboxy rhodol derivatives, tetramethyl and tetraethyl rhodamine, diphenyldimethyl and diphenyldiethyl rhodamine, dinaphthyl rhodamine, rhodamine 101 sulfonyl chloride, Cy3, Cy3B, Cy3.5, Cy5, Cy5.5, Cy7, DyLight650, IRDye650, IRDye680, DyLight750, Alexa Fluor 647, Alexa Fluor 750, IR800CW, ICG, Green Fluorescent Protein, EBFP, EBFP2, Azurite, mKalamal, ECFP, Cerulean, CyPet, YFP, Citrine, Venus, YPet, a gadolinium chelate, an iron oxide particle, a super paramagnetic iron oxide particle, an ultra-small paramagnetic particle, a manganese chelate, gallium containing agent, 64Cu diacetyl-bis(N4-methylthiosemicarbazone), 18F-fluorodeoxyglucose, 18F-fluoride, 3′-deoxy-3′-[18F]fluorothymidine, 18F-fluoromisonidazole, technetium-99m, thallium, iodine, barium-sulphate, or a combination thereof. In some embodiments, the method further comprises imaging the macrophage network. In some embodiments, (a) and (b) are performed substantially simultaneously. In some embodiments, (a) and (b) are performed sequentially. In some embodiments, the method further comprises, prior to (a), imaging the macrophage network and measuring a first signal. In some embodiments, the method further comprises, between (a) and (b), imaging the macrophage network and measuring a second signal. In some embodiments, (b) is performed if the second signal is reduced relative to the first signal. In some embodiments, the compound is less than about 20 kDa, less than about 15 kDa, less than about 10 kDa, less than about 5 kDa, less than about 4 kDa, less than about 3 kDa, less than about 2 kDa, less than about 1 kDa, or less than about 0.5 kDa in size.

In some aspects, provided herein, is a method of treating a granulomatous disease in a subject comprising: (a) administering a therapeutically effective amount of a compound capable of disrupting a macrophage network; and (b) administering a therapeutically effective amount of a therapeutic. In some embodiments, the therapeutic is an antibacterial therapeutic. In some embodiments, the antibacterial therapeutic comprises an antibiotic. In some embodiments, the antibiotic is isoniazid, rifampin, ethambutol, purazinamide, amikacin, kanamycin, or capreomycin. In some embodiments, the therapeutic is an anti-inflammatory therapeutic. In some embodiments, the anti-inflammatory therapeutic comprises a nonsteroidal anti-inflammatory drug (NSAID), a glucocorticoid, or a disease-modifying agent of rheumatoid diseases (DMARD). In some embodiments, the NSAID is asprin, celecoxib, diclofenac, diflunisal, etodolac, ibuprofen, indomethacin, ketoprofin, ketorolac, nabumetone, naproxen, oxaprozin, piroxicam, salsalate, sulindac, or tolmetin. In some embodiments, the glucocorticoid is beclomethasone, betamethasone, budesonide, cortisone, dexamethasone, hydrocortisone, methylprednisolone, prednisolone, prednisone, or triamcinolone. In some embodiments, the DMARD is methotrexate, sulfasalazine, hydroxychloroquinine, leflunomide, azathioprine, cyclosporine, etanercept, adalimumab, infliximab, certolizumab pegol, or golimumab.

In some aspects, provided herein, is a method of treating cancer in a subject comprising: (a) administering a therapeutically effective amount of a compound capable of disrupting a macrophage network; and (b) administering a therapeutically effective amount of an anti-cancer therapy. In some embodiments, the anti-cancer therapy is surgery, radiation therapy, immunotherapy, chemotherapy, targeted therapy, hormone therapy, or oncolytic viral therapy. In some embodiments, the immunotherapy comprises treatment with an antibody, an antibody-drug conjugate, an antibody-like molecule, an antibody fragment, a recombinant protein, a T-cell receptor, a T-cell, a cancer vaccine a cytokine, or Bacillus Calmette-Guérin (BCG). In some embodiments, the T-cell is a CAR T-cell. In some embodiments, the chemotherapy comprises treatment with a chemotherapeutic agent. In some embodiments, the hormone therapy comprises treatment with tamoxifen, toremifene, fulvestrant, letrozole, anastrozole, exemestane, leuprolide, goserelin, triptorelin, or histrelin. In some embodiments, the macrophage network comprises one or more tumor associated macrophages. In some embodiments, the compound is capable of killing the one or more macrophages. In some embodiments, the compound comprises a targeting moiety that targets the one or more macrophages. In some embodiments, the targeting moiety is a CD206 ligand. In some embodiments, the CD206 ligand comprises at least a portion of mannose, galactose, collagen, fucose, sulfated N-acetylgalactosamine, N-acetylglucosamine, luteinizing hormone, thyroid stimulating hormone, or a chondroitin sulfate. In some embodiments, the targeting moiety is a CD204 ligand. In some embodiments, the CD204 ligand comprises at least a portion of lipid A, oxidized low-density lipoprotein (LDL), acetylated LDL, malondialdehyde modified LDL, maleylated LDL, lysophasphatidylcholine, phophatidic acid, cholesterol, Apo A-I, Apo E, glycated type IV collagen, modified collagen type I, III and IV, biglycan, decorin, albumin, advanced glycation end product bovine serum albumin, b-amyloid fibrils, calreticulin, gp96, an HSP70 protein, a lipopolysaccharide, lymphotoxin-alpha, CpG DNA, calciprotein particles, a Neisseria meningitides surface protein, C reactive protein, hepatitis C virus NS3 protein, or Tamm-Horsfall protein. In some embodiments, the targeting moiety is a CD163 ligand. In some embodiments, the CD163 ligand comprises at least a portion of haptoglobin, hemoglobin, tumor necrosis factor-α (TNF-α)-like weak inducer of the apoptosis (TWEAK), gram positive and gram negative bacteria, or casein kinase II subunit beta. In some embodiments, the compound comprises a cytotoxic agent. In some embodiments, the cytotoxic agent is a chemotherapeutic agent, an anti-tubulin agent, a DNA modifying agent, or a small interfering ribonucleic acid. In some embodiments, the cytotoxic agent is selected from the group consisting of an auristatin, a dolastatin, auristatin E, Monomethyl auristatin E (MMAE), Monomethyl auristatin F (MMAF), dimethylvaline-valine-dolaisoleuine-dolaproine-phenylalanine-p-phenylenediamine (AFP), 5-benzoylvaleric acid-auristatin E ester (AEVB), auristatin EB (AEB), Ansamitocin, Mertansine/emtansine (DM1), ravtansine/soravtansine (DM4), Duocamycin, Calicheamicin, and pyrrolobenzodiazepines. In some embodiments, the compound comprises a structure of T-B-P, where T is the targeting moiety, B is a backbone, and P is the cytotoxic agent. In some embodiments, the cytotoxic agent is attached to the backbone via a cleavable linker L. In some embodiments, the cleavable linker is a disulfide bond. In some embodiments, the backbone is a peptide backbone. In some embodiments, the backbone is a dextran backbone. In some embodiments, the compound further comprises an imaging agent. In some embodiments, the imaging agent is 5-carboxyfluorescein, fluorescein-5-isothiocyanate, fluorescein-6-isothiocyanate, 6-carboxyfluorescein, tetramethylrhodamine-6-isothiocyanate, 5-carboxytetramethylrhodamine, 5-carboxy rhodol derivatives, tetramethyl and tetraethyl rhodamine, diphenyldimethyl and diphenyldiethyl rhodamine, dinaphthyl rhodamine, rhodamine 101 sulfonyl chloride, Cy3, Cy3B, Cy3.5, Cy5, Cy5.5, Cy7, DyLight650, IRDye650, IRDye680, DyLight750, Alexa Fluor 647, Alexa Fluor 750, IR800CW, ICG, Green Fluorescent Protein, EBFP, EBFP2, Azurite, mKalamal, ECFP, Cerulean, CyPet, YFP, Citrine, Venus, YPet, a gadolinium chelate, an iron oxide particle, a super paramagnetic iron oxide particle, an ultra-small paramagnetic particle, a manganese chelate, gallium containing agent, 64Cu diacetyl-bis(N4-methylthiosemicarbazone), 18F-fluorodeoxyglucose, 18F-fluoride, 3′-deoxy-3′-[18F]fluorothymidine, 18F-fluoromisonidazole, technetium-99m, thallium, iodine, barium-sulphate, or a combination thereof. In some embodiments, the method further comprises imaging the macrophage network. In some embodiments, (a) and (b) are performed substantially simultaneously. In some embodiments, (a) and (b) are preformed sequentially. In some embodiments, the method further comprises, prior to (a), imaging the macrophage network and measuring a first signal. In some embodiments, the method further comprises, between (a) and (b), imaging the macrophage network and measuring a second signal. In some embodiments, (b) is performed if the second signal is reduced relative to the first signal. In some embodiments, the compound is less than about 20 kDa, less than about 15 kDa, less than about 10 kDa, less than about 5 kDa, less than about 4 kDa, less than about 3 kDa, less than about 2 kDa, less than about 1 kDa, or less than about 0.5 kDa in size.

In some aspects, provided herein, is a composition comprising: (i) a targeting moiety capable of targeting a tumor associated macrophage and (ii) a cytotoxic agent, each attached to a peptide backbone, wherein the composition is less than 20 kDa in size. In some aspects, provided herein, is a composition comprising: (i) a targeting moiety capable of targeting a tumor associated macrophage and (ii) a cytotoxic agent, each attached to a dextran backbone, wherein the composition is less than 20 kDa in size. In some embodiments, the cytotoxic agent is attached to the peptide backbone via a linker. In some embodiments, the cytotoxic agent is attached to the dextran backbone via a linker. In some embodiments, the linker comprises a disulfide bond. In some embodiments, the targeting moiety is a CD206 ligand. In some embodiments, the CD206 ligand comprises at least a portion of mannose, galactose, collagen, fucose, sulfated N-acetylgalactosamine, N-acetylglucosamine, luteinizing hormone, thyroid stimulating hormone, or a chondroitin sulfate. In some embodiments, the targeting moiety is a CD204 ligand. In some embodiments, the CD204 ligand comprises at least a portion of lipid A, oxidized low-density lipoprotein (LDL), acetylated LDL, malondialdehyde modified LDL, maleylated LDL, lysophasphatidylcholine, phophatidic acid, cholesterol, Apo A-I, Apo E, glycated type IV collagen, modified collagen type I, III and IV, biglycan, decorin, albumin, advanced glycation end product bovine serum albumin, b-amyloid fibrils, calreticulin, gp96, an HSP70 protein, a lipopolysaccharide, lymphotoxin-alpha, CpG DNA, calciprotein particles, a Neisseria meningitides surface protein, C reactive protein, hepatitis C virus NS3 protein, or Tamm-Horsfall protein. In some embodiments, the targeting moiety is a CD163 ligand. In some embodiments, the CD163 ligand comprises at least a portion of haptoglobin, hemoglobin, tumor necrosis factor-α (TNF-α)-like weak inducer of the apoptosis (TWEAK), gram positive and gram negative bacteria, or casein kinase II subunit beta. In some embodiments, the cytotoxic agent is a chemotherapeutic agent, an anti-tubulin agent, a DNA modifying agent, or a small interfering ribonucleic acid. In some embodiments, the cytotoxic agent is selected from the group consisting of an auristatin, a dolastatin, auristatin E, Monomethyl auristatin E (MMAE), Monomethyl auristatin F (MMAF), dimethylvaline-valine-dolaisoleuine-dolaproine-phenylalanine-p-phenylenediamine (AFP), 5-benzoylvaleric acid-auristatin E ester (AEVB), auristatin EB (AEB), Ansamitocin, Mertansine/emtansine (DM1), ravtansine/soravtansine (DM4), Duocamycin, Calicheamicin, and pyrrolobenzodiazepines. In some aspects, provided herein, is a method of treating cancer comprising administering a therapeutically effective amount of the composition described herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A shows imaging of a tumor using Hoechst nuclear stain.

FIG. 1B shows imaging of a tumor using 3,000 molecular weight (MW) dextran dye.

FIG. 1C shows imaging of a tumor using fluorescent antibodies specific for the blood vessel marker CD31.

FIG. 1D shows imaging of a tumor using fluorescent antibodies specific for the macrophage marker CD206.

FIG. 1E shows imaging of a tumor using 10,000 MW dextran dye.

FIG. 2A shows time-course imaging of macrophages and HEK293s cells at 0 minute. Blue: nuclear stain (Hoechst), Green: dextran stain (pHrodo™ Green Dextran).

FIG. 2B shows time-course imaging of macrophages and HEK293s cells at 28 minutes. Blue: nuclear stain (Hoechst), Green: dextran stain (pHrodo™ Green Dextran).

FIG. 2C shows time-course imaging of macrophages and HEK293s cells at 17 hours. Blue: nuclear stain (Hoechst), Green: dextran stain (pHrodo™ Green Dextran).

FIG. 3A depicts a graph showing the analysis results of the time-course imaging of macrophages and HEK293s cells with TC-171-032 FITC. Red bars: macrophages, Blue bars: HEK293s cells.

FIG. 3B depicts a graph showing the analysis results of the time-course imaging of macrophages and HEK293s cells with TC-171-033 FITC. Red bars: macrophages, Blue bars: HEK293s cells.

FIG. 4A shows imaging of U87MG-tumor-bearing mouse brain (upper) and normal brain (lower). Blue: cell nuclei stained with Hoechst, Red: two-million MW rhodamine-dextran.

FIG. 4B shows imaging of U87MG-tumor-bearing mouse brain (upper) and normal brain (lower). Blue: cell nuclei stained with Hoechst, Red: 10,000 MW rhodamine-dextran.

FIG. 5A shows imaging of human glioblastoma in mouse brain. Cell Nuclei: Hoechst, CD206 targeting compound: pHrodo™ Green Dextran, Blood vessels CD31: fluorescent antibodies specific for the blood vessel marker CD31, and CD206 tumor associated macrophages: fluorescent antibodies specific for the macrophage marker Iba1.

FIG. 5B shows imaging of subcutaneous melanoma in mouse. Cell Nuclei: Hoechst, CD206 targeting compound: pHrodo™ Green Dextran, and CD206 tumor associated macrophages: fluorescent antibodies specific for the macrophage marker Iba1.

FIGS. 6A-6D shows CD163-expressing tumor associated macrophages in human malignant and benign meningioma. (FIG. 6A) A representative tumor tissue resected from a 56-year-old male with malignant meningioma. A CD163+ interconnected macrophage network is present in malignant meningioma. Hematoxylin stain (light grey) and CD163 immunoperoxidase (dark grey). (FIGS. 6B-6D) A representative tissue resected from a 44-year-old female with a benign (grade I) meningioma. (FIG. 6B) An Imaris three-dimensional (3D) rendition of anti-CD163 immunoperoxidase stained tissue from benign meningioma. Light gray colored structures depict CD163-expressing tumor associated macrophages and dark gray colored structures depict cell nuclei. (FIG. 6C) An enlarged view of the square in FIG. 6B showing a vascular mimicry channel formed by CD163-expressing tumor associated macrophages. (FIG. 6D) A cross-sectional view of the lumen associated with a CD163-expressing-macrophage-lined vascular mimicry tubular structure.

FIGS. 7A-7D shows that macrophages can form perfuse vascular mimicry tubular networks in low oxygen environments. (FIG. 7A) A confocal z-stack image of a macrophage 3D tubular network in experimental mice. (FIG. 7B) An Imaris 3D representation of the image in FIG. 7A. (FIG. 7C) The image in FIG. 7B made transparent to show that the intravenously-injected-fluorescent-dextran can circulate within the vascular mimicry tubes. (FIG. 7D) An enlarged view of the doted-square in FIG. 7C. The image in FIG. 7C was rotated along the horizontal axis to show that the macrophage vascular mimicry channels are perfused with the intravenously injected fluorescent dextran.

FIGS. 8A-8D shows that macrophages can form a granuloma-like structure. (FIG. 8A) A macrophage network forming a granuloma-like structure. (FIG. 8B) The macrophage network in FIG. 8A encasing a B16F10 uveal melanoma. (FIG. 8C) A macrophage network perfused with an intravenously injected 3,000 MW rhodamine-dextran. (FIG. 8D) The image of FIG. 8C made transparent with Imaris 3D imaging software.

### DETAILED DESCRIPTION OF THE INVENTION

Existing therapies for cancer or granulomatous diseases often demonstrate limited efficacy. The present application describes improved methods and compositions for treating cancer or granulomatous diseases that involve targeting and/or disrupting a macrophage network. Macrophages assemble into networks of channels that form a honeycomb-like structure around a tumor. Without wishing to be bound by any particular theory, it is thought that these networks (1) act as a shield around the tumor, preventing a chemotherapeutic, immunotherapeutic, or cell-based therapy from accessing the tumor, and/or (2) provide nutrients needed for continued growth of the tumor. Disclosed herein, in certain embodiments, are improved methods of treating cancer diseases comprising disrupting or destroying a macrophage network surrounding a tumor in order to (1) allow a therapeutic agent to access the tumor, (2) starve the tumor of nutrients that promote growth, and/or (3) provide targeted delivery of a therapeutic agent to the tumor. Accordingly, in some embodiments, there are provided herein methods of treating cancer comprising delivery of an agent capable of killing one or more macrophage cells of a macrophage network. In some embodiments, methods of treating cancer as disclosed herein comprise delivery of an agent to a tumor capable of perfusing through a macrophage network. In some embodiments, methods of treating cancer further comprise treatment with anti-cancer therapy (e.g., chemotherapy, immunotherapy, etc.).

Furthermore, neovascular diseases, including brain tumors such as glioblastoma, are characterized by a robust inflammatory infiltrate of macrophages. This population constitutes 30-50% of the cells in many solid tumors, including glioblastoma. Macrophages invade tumors and secrete various cytokines that promote remodeling of the extracellular matrix, neovascularization, and tumor growth. As provided herein, macrophages interconnect to form primitive channels that are distinct from blood vessels when exposed to hypoxic microenvironments including that found in tumors, in a process called vascular mimicry. Vascular mimicry serves as an alternate microcirculation that provides nourishment to tumors and was believed to be a process solely driven by cancer stem cells and not macrophages. Macrophages can also form a network of interconnected cells through the extension and fusion of cellular processes that emanate from their cell bodies (somas) and extend outwards to neighboring cells.

Most of the myeloid cells in central nervous system (CNS) tumors derive from systemic-monocyte-precursor cells that differentiate into macrophages in the tumor stroma. These macrophages express CD163, CD204, and CD206. Furthermore, in subcutaneous melanoma models, A375 (human) and B16F10 (mouse), CD163⁺, CD204⁺, and CD206⁺ macrophages are the predominant myeloid cell population associated with subcutaneous melanoma.

Additionally, macrophage networks are thought to form within or around a granuloma in the context of a granulomatous disease, such as an infectious disease (e.g., tuberculosis), thereby preventing a therapeutic agent (e.g., antibiotic) from accessing the infectious agent. Disclosed herein, in further embodiments, are improved methods of treating granulomatous diseases comprising disrupting or destroying a macrophage network within or around a granuloma in order to (1) allow a therapeutic to access the infectious agent and/or (2) provide targeted delivery of a therapeutic to the infectious agent. Disclosed herein, in certain embodiments, are methods of treating tuberculosis and other granulomatous diseases comprising disrupting a macrophage network. In some embodiments, methods of treating a granulomatous disease further comprise treatment with a therapeutic agent (e.g., an antibacterial agent).

Disclosed herein, in some embodiments, are compounds capable of targeting a macrophage of a macrophage network associated with a tumor or granuloma. In some embodiments, such a compound comprises: a targeting moiety and a cytotoxic agent, each attached to a backbone molecule (e.g., a peptide backbone or a dextran backbone). In some embodiments, compounds of the present disclosure are useful in, for example, disrupting a macrophage network or delivering an agent to a tumor or granuloma.

### Definitions

As used herein, a “macrophage network” generally refers to one or more macrophages and/or macrophage precursors (e.g., monocytes) that form a channel or network of channels. In some embodiments, a macrophage network is formed from macrophages, monocytes, or both, and/or myeloid precursor cells expressing both myeloid and stem cell markers. In some embodiments, a channel of a macrophage network comprises macrophages assembled into a channel structure. In some embodiments, a channel of a macrophage network comprises extracellular matrix and/or interstitial space which is remodeled into a channel structure by surrounding macrophages. In some embodiments, macrophages may form a network of interconnected cells through the extension and fusion of cellular processes that emanate from their cell bodies (somas) and extend outwards to neighboring cells. In some embodiments, a macrophage network comprises macrophages expressing CD206. In some embodiments, a macrophage network comprises macrophages expressing CD204. In some embodiments, a macrophage network comprises macrophages expressing both CD206 and CD204. In some embodiments, a macrophage network comprises macrophages expressing CD163. In some embodiments, cells within a macrophage network express CD206, CD204, and CD163. In some embodiments, a macrophage network comprises tumor associated macrophages. In some embodiments, a macrophage network is generated on or around cancer cells, for example, a tumor. In some embodiments, the macrophage network surrounds a tumor. In some embodiments, a macrophage network comprises macrophages which express markers that are not expressed on microglia. In some embodiments, a macrophage network is generated in the context of a granuloma. In some embodiments, the macrophage network surrounds the granuloma. In some embodiments, a macrophage network is a vascular mimicry network. In some embodiments, a macrophage network is a tubular network. In some embodiments, a macrophage network is capable of transporting molecules. In some embodiments, a macrophage network is capable of transporting nutrients to a tumor.

As used herein, the term “tumor associated macrophage” (TAM) generally refers to macrophages that exist in the microenvironment of a cancer, for example, a tumor. In some embodiments, a macrophage network comprises one or more TAMs.

As used herein, the phrase “disrupting a macrophage network” generally refers to reducing, eliminating, or otherwise modifying the structure of a macrophage network so that its function is impaired. In some embodiments, disrupting a macrophage network comprises killing one or more cells that make up a macrophage network. In some embodiments, disrupting a macrophage network comprises reducing the association between two or more cells that make up a macrophage network. In some embodiments, the disrupting of the macrophage network is sufficient to allow a therapeutic agent to access the tumor. In some embodiments, the disrupting of the macrophage network is sufficient to allow immune cells (e.g., T-cells) to access the tumor. In some embodiments, the disrupting of the macrophage network is sufficient to allow a therapeutic agent to access an infectious agent. In some embodiments, the disrupting of the macrophage network is sufficient to allow immune cells (e.g., T-cells) to access the infectious agent. In some embodiments, the disrupting of the macrophage network is sufficient to starve the tumor of nutrients that promote growth.

As used herein, the term “payload” generally refers to an agent delivered by a drug compound as disclosed herein. In some embodiments, it is a cytotoxic agent. In some embodiments, it is a macrophage polarizing agent. In some embodiments, it is an imaging agent.

As used herein, the term “subject” is used to mean any animal, preferably a mammal, including a human or non-human. The terms patient, subject, and individual are used interchangeably. None of the terms are to be interpreted as requiring the supervision of a medical professional (e.g., a doctor, nurse, physician's assistant, orderly, hospice worker).

The terms “treat,” “treating” or “treatment,” as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically. In some embodiments, treating a cancer includes preventing the growth of a tumor. In some embodiments, treating a cancer includes decreasing the size of a tumor. In some embodiments, treating a cancer includes eliminating a tumor. In some embodiments, treating an infectious disease includes preventing the spread of an infectious agent. In some embodiments, treating an infectious disease includes reducing the amount of an infectious agent in a subject. In some embodiments, treating an infectious disease includes eliminating an infectious agent from a subject. In some embodiments, treating an inflammatory disease comprises reducing the level of inflammation in a subject.

The term “about” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclosed methods. Further, “about” can mean plus or minus less than 1 or 1, 2, 3, 4, 5, 6, 7, 8, 9, 10, 11, 12, 13, 14, 15, 16, 17, 18, 19, 20, 25, 30, or greater than 30 percent, depending upon the situation and known or knowable by one skilled in the art. About also includes the exact amount. Hence “about 10 kDa” means “about 10 kDa” and also “10 kDa”.

### Disrupting a Macrophage Network

As disclosed herein, a macrophage network comprises one or more macrophages and/or macrophage precursors (e.g., monocytes) that form a channel or network of channels. In some embodiments, macrophages can also engage in vascular mimicry by forming a network of interconnected cells through the extension and fusion of cellular processes that emanate from their cell bodies (somas) and extend outwards to neighboring cells. In some embodiments, macrophages can form a conduit system in the interstitial space within the tumor stroma by remodeling the extracellular matrix (ECM) through their protease activities on ECM-proteins. A macrophage network is often capable of transporting molecules within the interior of the channel network. In some embodiments, the dimensions of the channels of a macrophage network limit the size of molecules which can perfuse the network and thereby be transported. In some embodiments, a macrophage network is capable of transporting molecules less than about 50 kDa, about 40 kDa, about 30 kDa, 20 kDa, about 15 kDa, about 10 kDa, about 5 kDa, about 4 kDa, about 3 kDa, about 2 kDa, about 1 kDa, or about 0.5 kDa in size, or smaller. In some embodiments, a macrophage network is incapable of transporting molecules greater than about 50 kDa, about 40 kDa, about 30 kDa 20 kDa, about 15 kDa, about 10 kDa, about 5 kDa, about 4 kDa, about 3 kDa, about 2 kDa, about 1 kDa, or about 0.5 kDa in size.

In some embodiments, a macrophage network is generated in the context of cancer, for example, a tumor. In some embodiments, a macrophage network surrounds a tumor. In some embodiments, a macrophage network is formed within a tumor (e.g., between cancer cells of a tumor). In some embodiments, a macrophage network comprises one or more tumor associated macrophages. In some embodiments, a macrophage network is generated in the context of a granulomatous disease, for example, an infectious disease or an inflammatory disease. In some embodiments, the macrophage network surrounds the granuloma. In some embodiments, a macrophage network can form in low oxygen environments. Cells which make up a macrophage network express one or more markers. In some embodiments, cells within a macrophage network express CD206. In some embodiments, cells within a macrophage network express CD204. In some embodiments, cells within a macrophage network express both CD206 and CD204. In some embodiments, cells within a macrophage network express CD163. In some embodiments, cells within a macrophage network express CD206, CD204, and CD163. In some embodiments, cells within a macrophage network express markers that are not expressed on microglia.

In some embodiments, the described methods comprise disrupting a macrophage network. In some embodiments, disrupting a macrophage network comprises killing one or more cells making up a macrophage network. In some embodiments, disrupting a macrophage network comprises killing one or more tumor associated macrophages, for example by specifically directing a cytotoxic agent to a tumor associated macrophage via a targeting moiety. In some embodiments, the disrupting of the macrophage network is sufficient to allow a therapeutic agent to access the tumor. In some embodiments, the disrupting of the macrophage network is sufficient to allow immune cells (e.g., T-cells) to access the tumor. Disrupting a macrophage network by killing one or more tumor associated macrophages is useful in, for example, increasing the effectiveness of anti-cancer therapy. In some embodiments, disrupting a macrophage network comprises killing one or more macrophages within a granuloma, for example by specifically directing a cytotoxic agent to a macrophage within a granuloma via a targeting moiety. In some embodiments, the disrupting of the macrophage network is sufficient to allow a therapeutic agent to access an infectious agent. In some embodiments, disrupting a macrophage network comprises killing one or more macrophages within a granuloma and, subsequently or simultaneously, providing a therapeutic agent capable of eliminating an infectious agent (e.g., tuberculosis). In some embodiments, the disrupting of the macrophage network is sufficient to allow immune cells (e.g., T-cells) to access the infectious agent. In some embodiments, disrupting a macrophage network comprises disrupting the association between cells of a macrophage network. In some embodiments, disrupting a macrophage network comprises modifying the polarization of a cell of a macrophage network, for example, by specifically directing a macrophage polarizing agent to a macrophage network. In some embodiments, a compound or agent is directed to a macrophage network by the use of a targeting agent. A targeting agent may be useful in, for example, preferentially directing a compound or agent to a macrophage network. For example, a targeting agent (e.g., a CD206 targeting agent, a CD204 targeting agent, or a CD163 targeting agent) may direct a compound to a macrophage network but not to other macrophages, such as microglia. Preferential targeting may serve to reduce or prevent toxicity from a compound outside of a macrophage network.

### Compounds

Described herein are compounds capable of targeting macrophages of a macrophage network (e.g., tumor associated macrophages). Such compounds are conjugate molecules comprising a targeting moiety that directs the compound to the macrophage coupled via a backbone molecule and/or a linker to an agent to be delivered to the macrophage. In some embodiments, the compound comprises a targeting moiety (e.g., a CD206 ligand, a CD204 ligand, or a CD163 ligand) and a therapeutic or cytotoxic agent (e.g., an anti-cancer therapeutic). In some embodiments, a compound comprises a targeting moiety and an imaging agent. In some embodiments, a compound further comprises a backbone molecule. In some embodiments, the backbone is a peptide backbone. In some embodiments, the backbone molecule is a polymer. In some embodiments, the backbone is a dextran molecule.

In some embodiments, the compound further comprises a linker. In some embodiments, a linker attaches an agent or moiety (e.g., a targeting moiety, an imaging agent, a therapeutic or cytotoxic agent) to the backbone. In some embodiments, the linker attaches the therapeutic or cytotoxic agent to the backbone. In some embodiments, the linker attaches an anti-cancer therapeutic to the backbone. In some embodiments, a linker is a non-cleavable linker. In some embodiments, a linker is a cleavable linker. In some embodiments, a cleavable linker is capable of being cleaved by an enzyme (e.g., a protease), a change in temperature, a change in pH, a chemical stimulus, or any combination thereof. In some embodiments, the cleavable linker comprises a disulfide bond. In some embodiments, the cleavable linker comprises a protease cleavage site. In some embodiments, the cleavable linker is capable of being cleaved by a lysosomal protease or an endosomal protease.

In some embodiments, a compound comprises a structure of T-B-P, where T is a targeting moiety, B is a backbone, and P is a therapeutic agent. In some embodiments the therapeutic agent is a cytotoxic agent. In some embodiments the T-B-P molecule further comprises a linker L. In some embodiments, the therapeutic agent P is attached to the backbone via the linker L, such that the structure of the compound is structure T-B-L-P. In some embodiments, the linker L is a cleavable linker. In some embodiments, the compound comprises MMAE and valine (Val)-Citrulline (Cit) as a cytotoxic agent and a linker, respectively. In some embodiments, a compound comprises an imaging agent I. In some embodiments, the structure of the compound is T-B-I. In some embodiments, the compound comprises a therapeutic agent P and an imaging agent I, such that the structure of the compound is T-B-P-I or T-B-L-P-I. In some embodiments, the imaging agent is attached to a linker. In some embodiments, the imaging agent is not attached to a linker.

Compounds of the present disclosure are useful in, for example, targeting macrophage cells of a macrophage network. In some embodiments, targeting cells of a macrophage network serves to identify the location of a macrophage network. For example, a compound is used to identify the location of a macrophage network surrounding a tumor, thereby determining the size and location of the tumor. In some embodiments, targeting cells of a macrophage network serves to disrupt a macrophage network, for example, by killing the cells (e.g., tumor associated macrophages) of a macrophage network with a cytotoxic agent. In some embodiments, targeting cells of a macrophage network serves to deliver an agent (e.g., a cytotoxic agent) to a tumor by entering and perfusing through the macrophage network. Targeting cells of a macrophage network may serve to deliver, for example, a cytotoxic agent, a chemotherapeutic agent, or an immunotherapeutic (e.g., PD1 targeting molecule, PDL1 targeting molecule, other immune checkpoint inhibitors). In some embodiments, a compound is capable of entering and perfusing through a macrophage network. In some embodiments, to be capable of entering and perfusing through a macrophage network, a compound must be of an appropriate size. In some embodiments, in order to be capable of entering and perfusing through a macrophage network, a compound is less than or equal to 50 kDa, less than or equal to 40 kDa, less than or equal to 30 kDa, less than or equal to 20 kDa, less than or equal to 10 kDa, less than or equal to 5 kDa, or smaller. In some embodiments, a compound is capable of entering and perfusing through a macrophage network and is unable to cross the blood-brain barrier. A compound capable of entering and perfusing through a macrophage network and incapable of crossing the blood-brain barrier may be useful in, for example, targeting a compound (e.g., a cytotoxic compound) to a macrophage network (e.g., a macrophage network around a tumor) while preventing the compound from accessing the brain cells of a subject. In some embodiments, a compound is capable of entering and perfusing a tumor through a macrophage network and is capable of crossing the blood-tumor barrier.

### Combination Therapy

Described herein are methods for treating a disease, for example, cancer or a granulomatous disease using a combination therapy. In some embodiments, combination therapy comprises disrupting a macrophage network and administering to a subject a therapeutically effective amount of a therapy. In some embodiments, disrupting a macrophage network and administering a therapeutically effective amount of a therapy are performed substantially simultaneously. In some embodiments, disrupting a macrophage network and administering a therapeutically effective amount of a therapy are performed sequentially. For example, a compound capable of disrupting a macrophage network is first provided to a subject, and then a therapeutically effective amount of a therapy is administered to a subject after the macrophage network has been disrupted, thereby treating the cancer or granulomatous disease. Combination therapy is useful in, for example, improving the efficacy of a therapy (e.g., an anti-cancer therapy) by first disrupting a macrophage network prior to, or simultaneous with, administering the therapy. In some embodiments, combination therapy comprises disrupting a macrophage network (e.g., by providing one or more compounds comprising a targeting agent as disclosed herein) and providing an anti-cancer therapy, thereby treating cancer in a patient. In some embodiments, an anti-cancer therapy is surgery, radiation, chemotherapy, immunotherapy, or a combination thereof.

In some embodiments, disrupting a macrophage network is useful in, for example, increasing the efficacy of an immunotherapy in treating cancer. In one example, a macrophage network around a tumor in a subject is disrupted using the methods and/or compounds disclosed herein, improving the accessibility of large molecules and immune cells (e.g., T-cells) to the tumor site and thereby improving the efficacy potential of an immunotherapy. In this example, an immunotherapy (e.g., immune checkpoint blockade, CAR T-cell therapy, adoptive T-cell therapy, etc.) is provided following disruption of the macrophage network, thereby treating the cancer in the subject.

In some embodiments, combination therapy further comprises imaging a macrophage network surrounding the cancer or granuloma to be treated. In some embodiments, combination therapy comprises: imaging a macrophage network and measuring a first signal, administering a compound capable of disrupting a macrophage network, imaging the macrophage network and measuring a second signal, comparing the second signal to the first signal, and administering a therapeutically effective amount of a therapy if the second signal is reduced relative to the first signal, indicating that the macrophage network has been disrupted. In some embodiments, a macrophage network is imaged in a subject suffering from cancer. In some embodiments, a macrophage network is imaged in a subject suffering from a granulomatous disease. In some embodiments, a macrophage network is imaged using an imaging agent directed to a macrophage network with a targeting agent. In some embodiments, the first signal is a fluorescent signal. In some embodiments, the compound comprises a targeting agent. In some embodiments, the compound comprises a cytotoxic agent. In some embodiments, the compound comprises a macrophage polarizing agent. In some embodiments, the compound is capable of entering and perfusing through a macrophage network. In some embodiments, the second signal is of the same type as the first signal. In some embodiments, a reduction in the second signal relative to the first signal indicates disruption of the macrophage network. In some embodiments, the therapy is an anti-cancer therapy. In some embodiments, the therapy is an immunotherapy. In some embodiments, the therapy is an antibacterial therapeutic. In some embodiments, the therapy is an anti-inflammatory therapeutic. In some embodiments, a therapy is administered if the second signal is reduced relative to the first signal.

In some embodiments, a method of treating cancer with combination therapy comprises: imaging a tumor and measuring a first signal, administering a compound capable of disrupting a macrophage network, imaging the tumor and measuring a second signal, comparing the second signal to the first signal, and administering a therapeutically effective amount of an anti-cancer therapy if the second signal is not increased or is decreased relative to the first signal. In some embodiments, the tumor is imaged using magnetic resonance imaging (MRI). In some embodiments, the tumor is imaged using positron emission tomography (PET) imaging. In some embodiments, the tumor is imaged using computed tomography (CT) imaging. In some embodiments, the compound comprises a targeting agent. In some embodiments, the compound comprises a cytotoxic agent. In some embodiments, the compound comprises a macrophage polarizing agent. In some embodiments, the compound is capable of entering and perfusing through a macrophage network. In some embodiments, the tumor is imaged by the same method as used before administering the compound. In some embodiments, the tumor is imaged by a different method from that used before administering the compound. In some embodiments, the second signal is compared to the first signal, thereby determining whether a tumor size has been reduced. In some embodiments, the anti-cancer therapy is administered is the second signal is reduced (e.g., is reduced in size or intensity) relative to the first signal.

### Cancer

As described herein, a macrophage network is capable of forming in the context of cancer, for example, around a tumor. In some embodiments, a macrophage network surrounds a tumor. In some embodiments, a tumor is a malignant tumor. In some embodiments, a tumor is a benign tumor. In some embodiments, a macrophage network around a tumor is disrupted, thereby treating the cancer. In some embodiments, the disrupting of the macrophage network around the tumor is sufficient to allow a therapeutic agent to access the tumor. In some embodiments, the disrupting of the macrophage network around the tumor is sufficient to allow immune cells (e.g., T-cells) to access the tumor. In some embodiments, the disrupting of the macrophage network around the tumor is sufficient to starve the tumor of nutrients that promote growth. In some embodiments, a cytotoxic agent is directed to a macrophage network using a targeting agent, thereby delivering the cytotoxic agent to a tumor. In some embodiments, a macrophage network around a tumor is disrupted, thereby improving the effectiveness of anti-cancer therapy in a subject. Examples of anti-cancer therapy are described elsewhere herein.

Examples of cancers that can be treated using the methods and compositions described herein include but are not limited to Adrenocortical Carcinoma, AIDS-Related Cancers, Anal Cancer, Astrocytoma, Basal Cell Carcinoma, Bile Duct Cancer, Bladder Cancer, Bladder Cancer, Bone Cancer, Brain Stem Glioma, Brain Tumor, Breast Cancer, Bronchial Adenomas, Carcinoid Tumor, Cerebellar Astrocytoma, Cervical Cancer, Colon Cancer, Colorectal Cancer, Endometrial Cancer, Ependymoma, Esophageal Cancer, Extragonadal Germ Cell Tumor, Intraocular Melanoma, Eye Cancer, Retinoblastoma, Gallbladder Cancer, Gastrointestinal Carcinoid Tumor, Gastrointestinal Stromal Tumor (GIST), Germ Cell Tumor (Extracranial), Germ Cell Tumor (Extragonadal), Germ Cell Tumor (Ovarian), Gestational Trophoblastic Tumor, Glioma, Head and Neck Cancer, Hypopharyngeal Cancer, Hypothalamic and Visual Pathway Glioma, Intraocular Melanoma, Islet Cell Carcinoma (Endocrine Pancreas), Kaposi's Sarcoma, Kidney (Renal Cell) Cancer, Laryngeal Cancer, Lip and Oral Cavity Cancer, Liver Cancer, Lung Cancer (Non-Small Cell), Lung Cancer (Small Cell), Malignant Fibrous Histiocytoma of Bone/Osteosarcoma, Medulloblastoma, Melanoma, Merkel Cell Carcinoma, Mesothelioma, Metastatic Squamous Neck Cancer with Occult Primary, Multiple Endocrine Neoplasia Syndrome, Multiple Myeloma/Plasma Cell Neoplasm, Mycosis Fungoides, Myelodysplastic Syndromes, Nasal Cavity and Paranasal Sinus Cancer, Nasopharyngeal Cancer, Neuroblastoma, Oral Cancer, Oropharyngeal Cancer, Ovarian Cancer, Ovarian Epithelial Cancer, Ovarian Germ Cell Tumor, Ovarian Low Malignant Potential Tumor, Pancreatic Cancer, Parathyroid Cancer, Penile Cancer, Pheochromocytoma, Pineoblastoma and Supratentorial Primitive Neuroectodermal Tumors, Pituitary Tumor, Plasma Cell Neoplasm/Multiple Myeloma, Pleuropulmonary Blastoma, Prostate Cancer, Rectal Cancer, Retinoblastoma, Rhabdomyosarcoma, Salivary Gland Cancer, Sarcoma (Kaposi's), Sarcoma (uterine), Sezary Syndrome, Skin Cancer (non-Melanoma), Skin Cancer (Melanoma), Skin Carcinoma (Merkel Cell), Small Intestine Cancer, Soft Tissue Sarcoma, Squamous Cell Carcinoma, Stomach (Gastric) Cancer, Testicular Cancer, Thymoma, Thyroid Cancer, Trophoblastic Tumor, Gestational, Urethral Cancer, Uterine Cancer, Endometrial, Uterine Sarcoma, Vaginal Cancer, Visual Pathway and Hypothalamic Glioma, Vulvar Cancer, Waldenstrom's Macroglobulinemia, Wilms' Tumor, and the like. In some embodiments, methods and compositions described herein are used to treat breast cancer, lung cancer, ovarian cancer, or renal cell cancer. In some embodiments, methods and compositions described herein are used to treat a Schwannoma, a Meningioma, or a Hemangioblastoma. In some embodiments, methods and compositions described herein are used to treat Melanoma, Glioblastoma, Medulloblastoma, Astrocytoma, or Neuroblastoma. In some embodiments, methods and compositions described herein are used to treat Glioma. In some embodiments, methods and compositions described herein are used to treat Melanoma. In some embodiments, methods and compositions described herein are used to treat Glioblastoma.

Granulomatous diseases.

As described herein, a macrophage network is capable of forming in the context of granuloma. In some embodiments, a granuloma is formed in a subject suffering from a granulomatous disease (e.g., an infectious disease or an inflammatory disease). In some embodiments, a granuloma is formed in a subject suffering from tuberculosis. A granuloma comprises one or more cells (e.g., macrophages). In some embodiments, a macrophage network is disrupted by targeting cells of a granuloma using a targeting agent. In some embodiments, disruption of a macrophage network improves the effectiveness of an antibacterial agent (e.g., an antibiotic). In some embodiments, disruption of a macrophage network improves the effectiveness of an anti-inflammatory agent.

In some embodiments, a granulomatous disease is an infectious disease. In some embodiments, an infectious disease is tuberculosis, histoplasmosis, cryptococcis, coccidiomycosis, leprosy, blastomycoccis, or cat scratch disease. In some embodiments, the granulomatous disease is tuberculosis. In some embodiments, a granulomatous disease is not an infectious disease. In some embodiments, a granulomatous disease is sarcoidosis, berylliosis, granulomatosis with polyangiitis, giant cell tumor disease, Rosai-Dorfman disease, rheumatoid arthritis, or Crohn's disease.

### Anti-Cancer Therapy

In some embodiments, the methods disclosed herein comprise use of anti-cancer therapy. In some embodiments, anti-cancer therapy is used in combination with disruption of a macrophage network (e.g., killing one or more tumor associated macrophages) to treat cancer in a subject. Examples of anti-cancer therapy include but are not limited to surgery, radiation therapy, immunotherapy, chemotherapy, targeted therapy, hormone therapy, and oncolytic viral therapy. In some embodiments immunotherapy comprises treatment with an antibody, an antibody-drug conjugate, an antibody-like molecule, an antibody fragment, a recombinant protein, a T-cell receptor, a T-cell (e.g., a chimeric antigen receptor T-cell), a cancer vaccine, a cytokine, or Bacillus Calmette-Guérin (BCG). In some embodiments, chemotherapy comprises treatment with a chemotherapeutic agent. In some embodiments, hormone therapy comprises treatment with tamoxifen, toremifene, fulvestrant, letrozole, anastrozole, exemestane, leuprolide, goserelin, triptorelin, or histrelin.

In some embodiments, disrupting a macrophage network using the methods and compositions of the present disclosure serves to increase the effectiveness of one or more anti-cancer therapies. In some embodiments, disrupting a macrophage network improves or increases accessibility of an anti-cancer therapy to the tumor. In some embodiments, disrupting a macrophage network improves the ability of immune cells (e.g., natural T-cells, engineered T-cells) to contact a tumor. In some embodiments, disrupting a macrophage network improves the ability of molecules that would otherwise be prevented from contacting a tumor (e.g., larger therapeutic agents such as antibodies, antibody fragments, recombinant proteins, antibody-drug conjugates, T-cell receptors, etc.) to contact the tumor. In some embodiments, improved tumor accessibility allows for increased efficacy of an anti-cancer therapy, for example, immunotherapy. In some embodiments, methods of the present disclosure comprise combination therapy comprising disrupting a macrophage network and providing an anti-cancer therapy, thereby treating cancer in a subject.

### Targeting Agents

In some embodiments, the methods disclosed herein comprise the use of a targeting agent (used interchangeably herein with “targeting moiety”) to target or otherwise direct a compound, agent, or other molecule to a macrophage network. In some embodiments, a targeting agent is an agent capable of binding to a protein on the surface of a macrophage of a macrophage network. In some embodiments, a protein is a cell surface receptor. In some embodiments, a cell is a macrophage. In some embodiments, a cell is a monocyte.

In some embodiments, a targeting agent is capable of binding to CD206. In some embodiments, a targeting agent comprises a CD206 ligand or portion thereof. In some embodiments, the targeting agent comprises at least a portion of mannose, galactose, collagen, fucose, sulfated N-acetylgalactosamine, N-acetylglucosamine, luteinizing hormone, thyroid stimulating hormone, or a chondroitin sulfate. In some embodiments, the targeting agent comprises mannose. In some embodiments, the targeting agent comprises galactose. In some embodiments, the targeting agent comprises collagen. In some embodiments, the targeting agent comprises fucose. In some embodiments, the targeting agent comprises N-acetylglucosamine. A targeting agent capable of binding to CD206 is able to target a cell of a macrophage network (e.g., a tumor associated macrophage) by binding to CD206 present on the surface of a cell.

Provided herein are new molecules identified by conducting Computer-Aided Drug Discovery (“CADD”) that can target the mannose and collagen binding sites of the CD206 receptor (Table 1 and Table 2). In some embodiments, the targeting agent or targeting moiety is a compound in Table 1 or Table 2. In some embodiments, the targeting agent or targeting moiety is a compound with a structure shown in Table 1 or Table 2. In some embodiments, the targeting agent or targeting moiety is EMOL_5581040_5581039. In some embodiments, the targeting agent or targeting moiety is EMOL_257460529_71962160. Although the current study focuses on targeting the mannose-binding site of the CD206 receptor, those skilled in the art would appreciate that multiple targeting moieties could be used for the CD206 receptor.

TABLE 1
Exemplary compounds that bind the collagen binding site of CD206

| | Compound | Site | STRUCTURE |
|---|---|---|---|
| 1 | Ac_SPG_Nme | 2_Collagen | 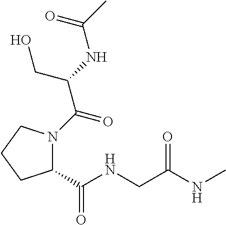 [SMILES: CNC(=O)CNC(=O)[C@@H]1CCCN1C(=O)[C@H](CO)NC(C)=O] |
| 2 | EMOL_5581040_ 5581039 | 2_Collagen | 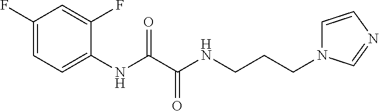 [SMILES: O=C(NCCCn1ccnc1)C(=O)Nc1ccc(F)cc1F] |
| 3 | EMOL_4663664_ 4663663 | 2_Collagen | 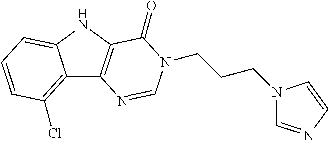 [SMILES: O=c1c2[nH]c3cccc(Cl)c3c2ncn1CCCn1ccnc1] |
| 4 | EMOL_101912707_ 101912706 | 2_Collagen | 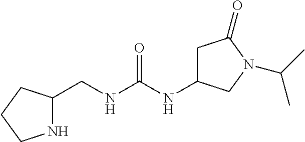 [SMILES: CC(C)N1CC(NC(=O)NCC2CCCN2)CC1=O] |
| 5 | EMOL_4685162_ 4685161 | 2_Collagen | 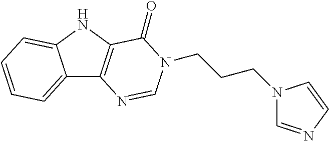 [SMILES: O=c1c2[nH]c3ccccc3c2ncn1CCCn1ccnc1] |
| 6 | EMOL_4677428_ 4677427 | 2_Collagen | 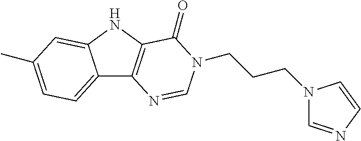 [SMILES: Cc1ccc2c(c1)[nH]c1c(=O)n(CCCn3ccnc3)cnc12] |
| 7 | EMOL_101917048_ 101917047 | 2_Collagen | 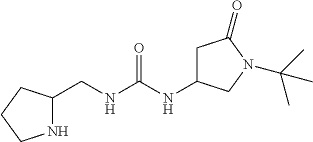 [SMILES: CC(C)(C)N1CC(NC(=O)NCC2CCCN2)CC1=O] |
| 8 | EMOL_101939883_ 101939882 | 2_Collagen | 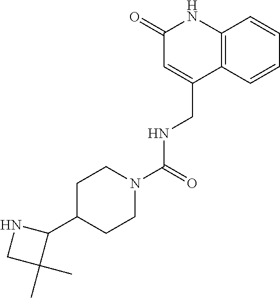 [SMILES: CC1(C)CNC1C1CCN(C(=O)NCc2cc(=O)[nH]c3ccccc23)CC1] |
| 9 | EMOL_102092393_ 102092392 | 2_Collagen | 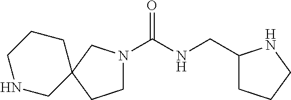 [SMILES: O=C(NCC1CCCN1)N1CCC2(CCCNC2)C1] |
| 10 | EMOL_102874923_ 102874922 | 2_Collagen | 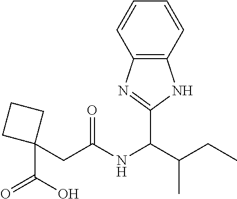 [SMILES: CCC(C)C(NC(=O)CC1(C(=O)O)CCC1)c1nc2ccccc2[nH]1] |
| 11 | EMOL_72193070_ 72193069 | 2_Collagen | 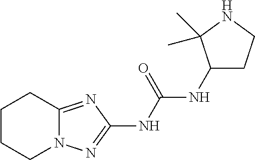 [SMILES: CC1(C)NCCC1NC(=O)Nc1nc2n(n1)CCCC2] |
| 12 | EMOL_101806242_ 101806241 | 2_Collagen | 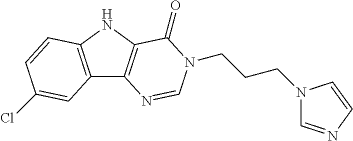 [SMILES: O=c1c2[nH]c3ccc(Cl)cc3c2ncn1CCCn1ccnc1] |
| 13 | EMOL_4798274_ 4798273 | 2_Collagen | 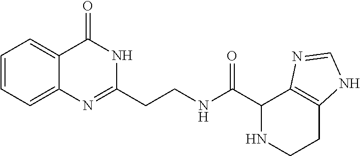 [SMILES: O=C(NCCc1nc2ccccc2c(=O)[nH]1)C1NCCc2[nH]cnc21] |
| 14 | EMOL_43147686_ 43147685 | 2_Collagen | 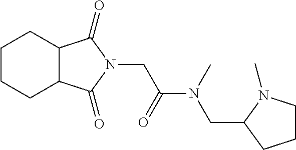 [SMILES: CN(CC1CCCN1C)C(=O)CN1C(=O)C2CCCCC2C1=O] |
| 15 | EMOL_102173522_ 102173521 | 2_Collagen | 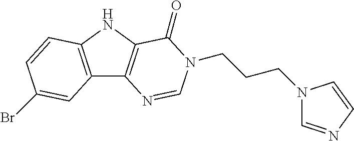 [SMILES: O=c1c2[nH]c3ccc(Br)cc3c2ncn1CCCn1ccnc1] |
| 16 | EMOL_4444831_ 4444830 | 2_Collagen | 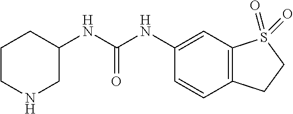 [SMILES: O=C(Nc1ccc2c(c1)S(=O)(=O)CC2)NC1CCCNC1] |
| 17 | EMOL_101758438_ 101758437 | 2_Collagen | 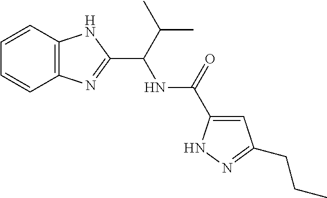 [SMILES: CCCc1cc(C(=O)NC(c2nc3ccccc3[nH]2)C(C)C)[nH]n1] |
| 18 | EMOL_18875602_ 18875601 | 2_Collagen | 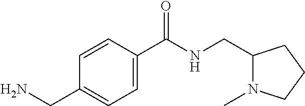 [SMILES: CN1CCCC1CNC(=O)c1ccc(CN)cc1] |
| 19 | EMOL_5496267_ 5496266 | 2_Collagen | 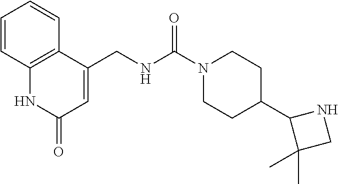 [SMILES: CC1(C)CNC1C1CCN(C(=O)NCc2cc(=O)[nH]c3ccccc23)CC1] |
| 20 | EMOL_257773321_ 51538120 | 2_Collagen | 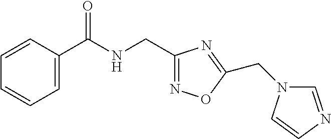 [SMILES: O=C(NCc1noc(Cn2ccnc2)n1)c1ccccc1] |
| 21 | EMOL_257193213_ 71585588 | 2_Collagen | 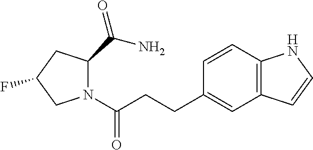 [SMILES: NC(=O)[C@@H]1C[C@@H](F)CN1C(=O)CCc1ccc2[nH]ccc2c1] |
| 22 | EMOL_25664883_ 25664882 | 2_Collagen | 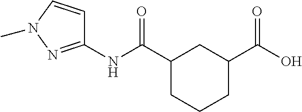 [SMILES: Cn1ccc(NC(=O)C2CCCC(C(=O)O)C2)n1] |
| 23 | EMOL_72963183_ 72963182 | 2_Collagen | 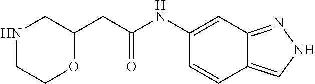 [SMILES: O=C(CC1CNCCO1)Nc1ccc2c[nH]nc2c1] |
| 24 | EMOL_177203137_ 177203136 | 2_Collagen | 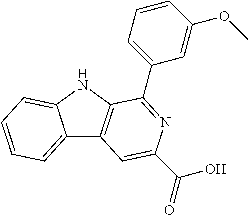 [SMILES: COc1cccc(-c2nc(C(=O)O)cc3c2[nH]c2ccccc23)c1] |
| 25 | EMOL_256874616_ 71237152 | 2_Collagen | 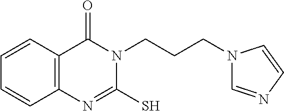 [SMILES: O=c1c2ccccc2nc(S)n1CCCn1ccnc1] |
| 26 | EMOL_17012212_ 17012211 | 2_Collagen | 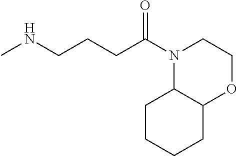 [SMILES: CNCCCC(=O)N1CCOC2CCCCC21] |
| 27 | EMOL_2008735_ 2008734 | 2_Collagen | 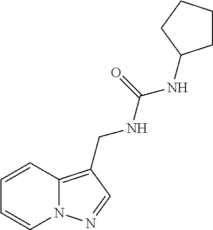 [SMILES: O=C(NCc1cnn2ccccc12)NC1CCCC1] |
| 28 | EMOL_37960573_ 37960572 | 2_Collagen | 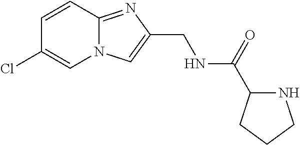 [SMILES: O=C(NCc1cn2cc(Cl)ccc2n1)C1CCCN1] |
| 29 | EMOL_43164674_ 43164673 | 2_Collagen | 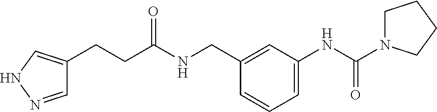 [SMILES: O=C(CCc1cn[nH]c1)NCc1cccc(NC(=O)N2CCCC2)c1] |
| 30 | EMOL_257763999_ 71523184 | 2_Collagen | 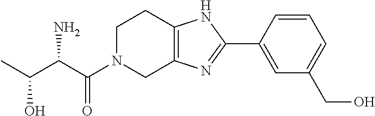 [SMILES: C[C@@H](O)[C@H](N)C(=O)N1CCc2[nH]c(-c3cccc(CO)c3)nc2C1] |
| 31 | EMOL_258037650_ 258037649 | 2_Collagen | 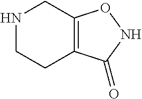 |
| 32 | EMOL_55606256_ 55606255 | 2_Collagen | 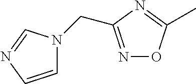 |
| 33 | EMOL_258091974_ 258091973 | 2_Collagen | 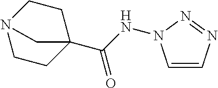 [SMILES: O=C(Nn1ccnn1)C12CCN(CC1)C2] |
| 34 | EMOL_594607_ 594606 | 2_Collagen | 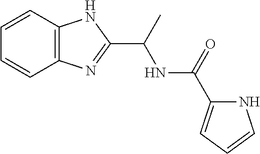 [SMILES: CC(NC(=O)c1ccc[nH]1)c1nc2ccccc2[nH]1] |
| 35 | EMOL_37977701_ 37977700 | 2_Collagen | 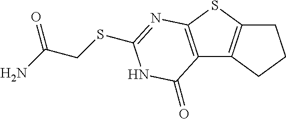 [SMILES: NC(=O)CSc1nc2sc3c(c2c(=O)[nH]1)CCC3] |
| 36 | EMOL_68893629_ 68893628 | 2_Collagen | 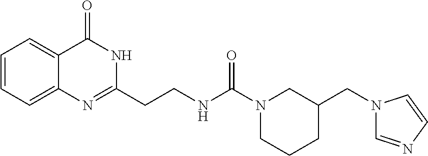 [SMILES: O=C(NCCc1nc2ccccc2c(=O)[nH]1)N1CCCC(Cn2ccnc2)C1] |
| 37 | EMOL_30695668_ 30695667 | 2_Collagen | 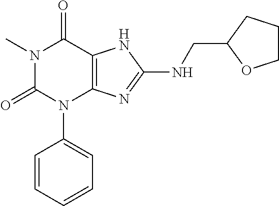 [SMILES: Cn1c(=O)c2[nH]c(NCC3CCCO3)nc2n(-c2ccccc2)c1=O] |
| 38 | EMOL_4393952_ 4393951 | 2_Collagen | 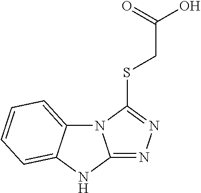 [SMILES: O=C(O)CSc1nnc2[nH]c3ccccc3n12] |
| 39 | EMOL_177494017_ 177494016 | 2_Collagen | 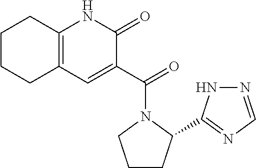 [SMILES: O=C(c1cc2c([nH]c1=O)CCCC2)N1CCC[C@H]1c1ncn[nH]1] |
| 40 | EMOL_2282546_ 2282545 | 2_Collagen | 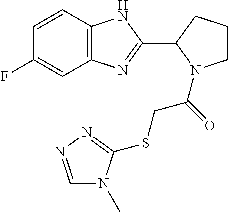 [SMILES: Cn1cnnc1SCC(=O)N1CCCC1c1nc2cc(F)ccc2[nH]1] |
| 41 | EMOL_43138072_ 43138071 | 2_Collagen | 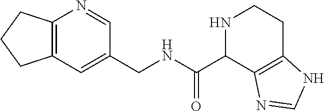 [SMILES: O=C(NCc1cnc2c(c1)CCC2)C1NCCc2[nH]cnc21] |
| 42 | EMOL_1478425_ 1478424 | 2_Collagen | 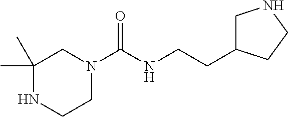 [SMILES: CC1(C)CN(C(=O)NCCC2CCNC2)CCN1] |
| 43 | EMOL_258027770_ 258027769 | 2_Collagen | 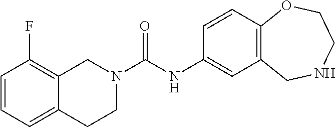 [SMILES: O=C(Nc1ccc2c(c1)CNCCO2)N1CCc2cccc(F)c2C1] |
| 44 | EMOL_68896827_ 68896826 | 2_Collagen | 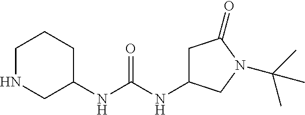 [SMILES: CC(C)(C)N1CC(NC(=O)NC2CCCNC2)CC1=O] |
| 45 | EMOL_5116807_ 5116806 | 2_Collagen | 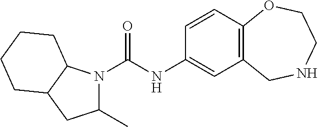 [SMILES: CC1CC2CCCCC2N1C(=O)Nc1ccc2c(c1)CNCCO2] |
| 46 | EMOL_102068372_ 102068371 | 2_Collagen | 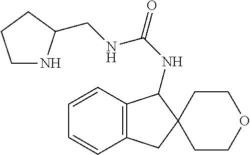 [SMILES: O=C(NCC1CCCN1)NC1c2ccccc2CC12CCOCC2] |
| 47 | EMOL_101832273_ 101832272 | 2_Collagen | 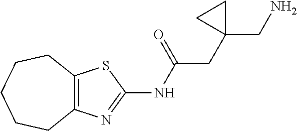 [SMILES: NCC1(CC(=O)Nc2nc3c(s2)CCCCC3)CC1] |
| 48 | EMOL_101916994_ 101916993 | 2_Collagen | 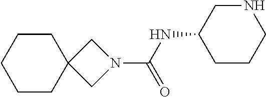 [SMILES: O=C(N[C@H]1CCCNC1)N1CC2(CCCCC2)C1] |
| 49 | EMOL_101830224_ 101830223 | 2_Collagen | 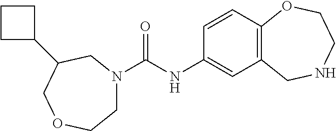 [SMILES: O=C(Nc1ccc2c(c1)CNCCO2)N1CCOCC(C2CCC2)C1] |
| 50 | EMOL_101875777_ 101875776 | 2_Collagen | 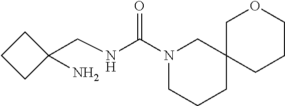 [SMILES: NC1(CNC(=O)N2CCCC3(CCCOC3)C2)CCC1] |
| 51 | EMOL_102122158_ 102122157 | 2_Collagen | 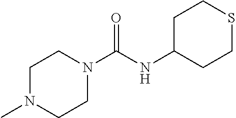 [SMILES: CN1CCN(C(=O)NC2CCSCC2)CC1] |
| 52 | EMOL_102871431_ 102871430 | 2_Collagen | 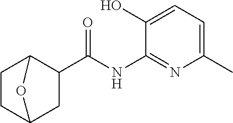 [SMILES: Cc1ccc(O)c(NC(=O)C2CC3CCC2O3)n1] |
| 53 | EMOL_101806983_ 101806982 | 2_Collagen | 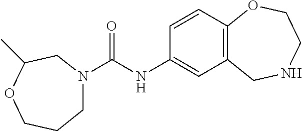 [SMILES: CC1CN(C(=O)Nc2ccc3c(c2)CNCCO3)CCCO1] |
| 54 | EMOL_102931778_ 102931777 | 2_Collagen | 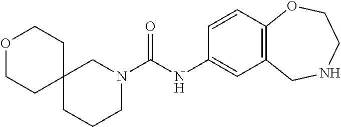 [SMILES: O=C(Nc1ccc2c(c1)CNCCO2)N1CCCC2(CCOCC2)C1] |
| 55 | EMOL_102367525_ 102367524 | 2_Collagen | 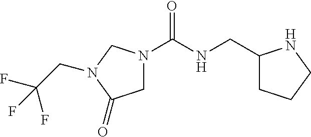 [SMILES: O=C1CN(C(=O)NCC2CCCN2)CN1CC(F)(F)F] |
| 56 | EMOL_101529921_ 101529920 | 2_Collagen | 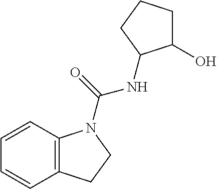 [SMILES: O=C(NC1CCCC1O)N1CCc2ccccc21] |
| 57 | EMOL_101822499_ 101822498 | 2_Collagen | 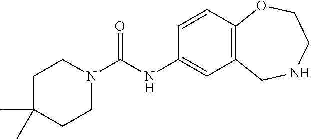 [SMILES: CC1(C)CCN(C(=O)Nc2ccc3c(c2)CNCCO3)CC1] |
| 58 | EMOL_101828451_ 101828450 | 2_Collagen | 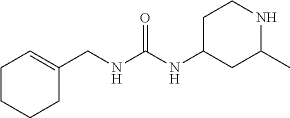 [SMILES: CC1CC(NC(=O)NCC2=CCCCC2)CCN1] |
| 59 | EMOL_102086927_ 102086926 | 2_Collagen | 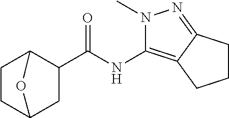 [SMILES: Cn1nc2c(c1NC(=O)C1CC3CCC1O3)CCC2] |
| 60 | EMOL_102805205_ 102805204 | 2_Collagen | 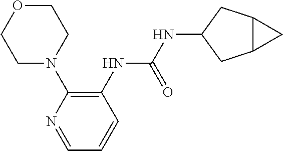 [SMILES: O=C(Nc1cccnc1N1CCOCC1)NC1CC2CC2C1] |
| 61 | EMOL_101822493_ 101822492 | 2_Collagen | 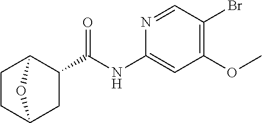 [SMILES: COc1cc(NC(=O)[C@@H]2C[C@H]3CC[C@@H]2O3)ncc1Br] |
| 62 | EMOL_101901652_ 101901651 | 2_Collagen | 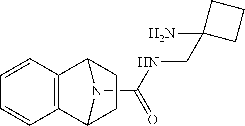 [SMILES: NC1(CNC(=O)N2C3CCC2c2ccccc23)CCC1] |
| 63 | EMOL_101533830_ 101533829 | 2_Collagen | 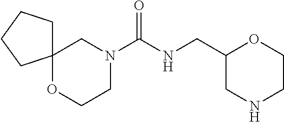 [SMILES: O=C(NCC1CNCCO1)N1CCOC2(CCCC2)C1] |
| 64 | EMOL_102191560_ 102191559 | 2_Collagen | 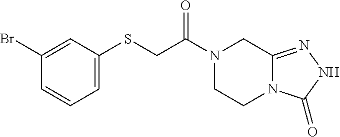 [SMILES: O=C(CSc1cccc(Br)c1)N1CCn2c(n[nH]c2=O)C1] |
| 65 | EMOL_101536392_ 101536391 | 2_Collagen | 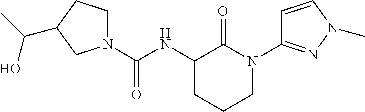 [SMILES: CC(O)C1CCN(C(=O)NC2CCCN(c3ccn(C)n3)C2=O)C1] |
| 66 | EMOL_102066674_ 102066673 | 2_Collagen | 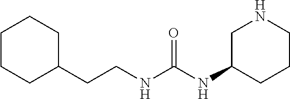 [SMILES: O=C(NCCC1CCCCC1)N[C@@H]1CCCNC1] |
| 67 | EMOL_101990483_ 101990482 | 2_Collagen | 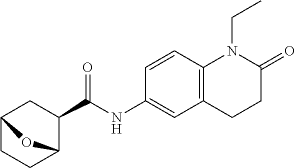 [SMILES: CCN1C(=O)CCc2cc(NC(=O)[C@@H]3C[C@H]4CC[C@@H]3O4)ccc21] |
| 68 | EMOL_101110374_ 101110373 | 2_Collagen | 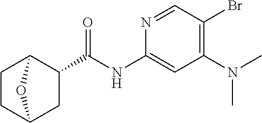 [SMILES: CN(C)c1cc(NC(=O)[C@@H]2C[C@H]3CC[C@@H]2O3)ncc1Br] |
| 69 | EMOL_111044370_ 1111044369 | 2_Collagen | 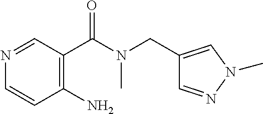 [SMILES: CN(Cc1cnn(C)c1)C(=O)c1cnccc1N] |
| 70 | EMOL_101867649_ 101867648 | 2_Collagen | 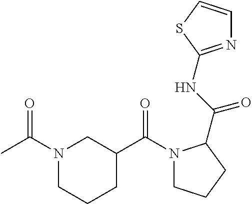 [SMILES: CC(=O)N1CCCC(C(=O)N2CCCC2C(=O)Nc2nccs2)C1] |
| 71 | EMOL_101534349_ 101534348 | 2_Collagen | 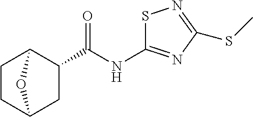 [SMILES: CSc1nsc(NC(=O)[C@@H]2C[C@H]3CC[C@@H]2O3)n1] |
| 72 | EMOL_101528637_ 101528636 | 2_Collagen | 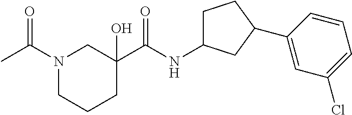 [SMILES: CC(=O)N1CCCC(O)(C(=O)NC2CCC(c3cccc(Cl)c3)C2)C1] |
| 73 | EMOL_257648588_ 52002095 | 2_Collagen | 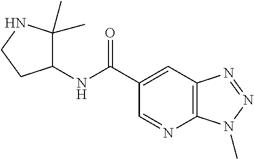 [SMILES: Cn1nnc2cc(C(=O)NC3CCNC3(C)C)cnc21] |
| 74 | EMOL_110721211_ 110721210 | 2_Collagen | 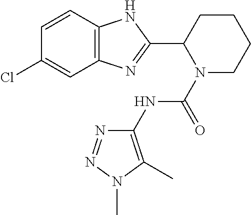 [SMILES: Cc1c(NC(=O)N2CCCCC2c2nc3cc(Cl)ccc3[nH]2)nnn1C] |
| 75 | EMOL_101733895_ 101733894 | 2_Collagen | 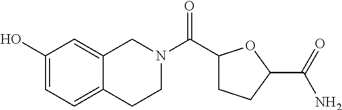 [SMILES: NC(=O)C1CCC(C(=O)N2CCc3ccc(O)cc3C2)O1] |
| 76 | EMOL_101502477_ 101502476 | 2_Collagen | 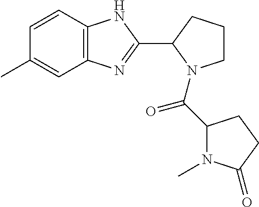 [SMILES: Cc1ccc2[nH]c(C3CCCN3C(=O)C3CCC(=O)N3C)nc2c1] |
| 77 | EMOL_102110567_ 102110566 | 2_Collagen | 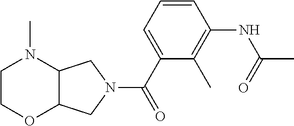 [SMILES: CC(=O)Nc1cccc(C(=O)N2CC3OCCN(C)C3C2)c1C] |
| 78 | EMOL_101930804_ 101930803 | 2_Collagen | 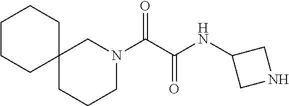 [SMILES: O=C(NC1CNC1)C(=O)N1CCCC2(CCCCC2)C1] |
| 79 | EMOL_110894315_ 110894314 | 2_Collagen | 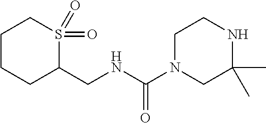 [SMILES: CC1(C)CN(C(=O)NCC2CCCCS2(=O)=O)CCN1] |
| 80 | EMOL_110909875_ 110909874 | 2_Collagen | 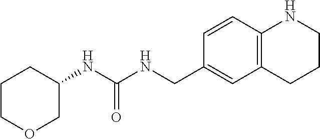 [SMILES: O=C(NCc1ccc2c(c1)CCCN2)N[C@H]1CCCOC1] |
| 81 | EMOL_111270428_ 111270427 | 2_Collagen | 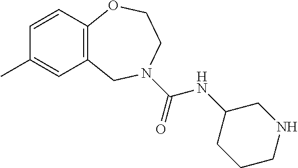 [SMILES: Cc1ccc2c(c1)CN(C(=O)NC1CCCNC1)CCO2] |
| 82 | EMOL_102880854_ 102880853 | 2_Collagen | 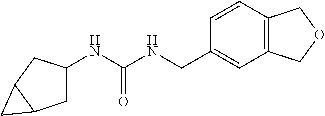 [SMILES: O=C(NCc1ccc2c(c1)COC2)NC1CC2CC2C1] |
| 83 | EMOL_101906527_ 101906526 | 2_Collagen | 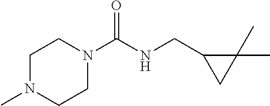 |
| 84 | EMOL_101891257_ 101891256 | 2_Collagen | 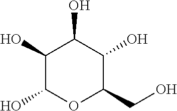 [SMILES: OC[C@H]1O[C@H](O)[C@@H](O)[C@@H](O)[C@@H]1O] |
| 85 | EMOL_102063038_ 102063037 | 2_Collagen | 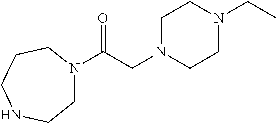 [SMILES: CCN1CCN(CC(=O)N2CCCNCC2)CC1] |
| 86 | EMOL_102198400_ 102198399 | 2_Collagen | 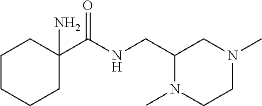 [SMILES: CN1CCN(C)C(CNC(=O)C2(N)CCCCC2)C1] |
| 87 | EMOL_102583894_ 102583893 | 2_Collagen | 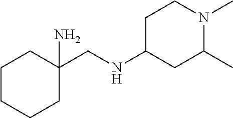 [SMILES: CC1CC(NCC2(N)CCCCC2)CCN1C] |

TABLE 2
Exemplary compounds that bind the mannose binding site of CD206

| | Compound | Site | STRUCTURE |
|---|---|---|---|
| 88 | Mannose | 3_Mannose | 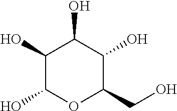 [SMILES: OC[C@H]1O[C@H](O)[C@@H](O)[C@@H](O)[C@@H]1O] |
| 89 | EMOL_257460529_ 71962160 | 3_Mannose | 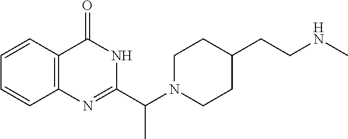 [SMILES: CNCCC1CCN(C(C)c2nc3ccccc3c(=O)[nH]2)CC1] |
| 90 | EMOL_42957666_ 42957665 | 3_Mannose | 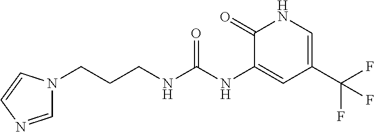 [SMILES: O=C(NCCCn1ccnc1)Nc1cc(C(F)(F)F)c[nH]c1=O] |
| 91 | EMOL_177317492_ 177317491 | 3_Mannose | 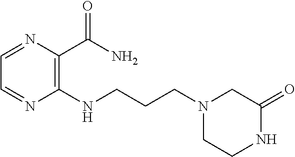 [SMILES: NC(=O)c1nccnc1NCCCN1CCNC(=O)C1] |
| 92 | EMOL_29918732_ 29918731 | 3_Mannose | 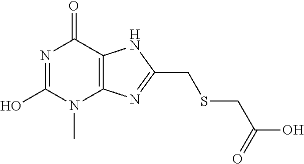 [SMILES: Cn1c(O)nc(=O)c2[nH]c(CSCC(=O)O)nc21] |
| 93 | EMOL_256904434_ 52591876 | 3_Mannose | 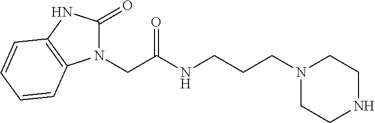 [SMILES: O=C(Cn1c(=O)[nH]c2ccccc21)NCCCN1CCNCC1] |
| 94 | EMOL_52489614_ 52489613 | 3_Mannose | 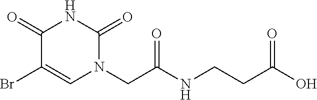 [SMILES: O=C(O)CCNC(=O)Cn1cc(Br)c(=O)[nH]c1=O] |
| 95 | EMOL_102067184_ 102067183 | 3_Mannose | 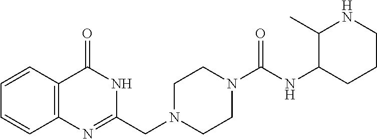 [SMILES: CC1NCCCC1NC(=O)N1CCN(Cc2nc3ccccc3c(=O)[nH]2)CC1] |
| 96 | EMOL_188864138_ 188864137 | 3_Mannose | 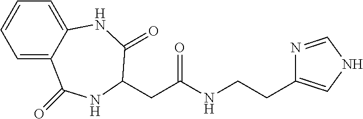 [SMILES: O=C(CC1NC(=O)c2ccccc2NC1=O)NCCc1c[nH]cn1] |
| 97 | EMOL_110194778_ 110194777 | 3_Mannose | 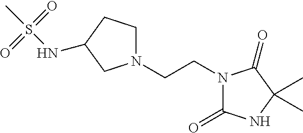 |
| 98 | EMOL_31933262_ 31933261 | 3_Mannose | 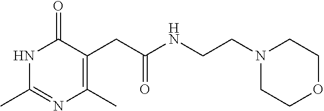 [SMILES: Cc1nc(C)c(CC(=O)NCCN2CCOCC2)c(=O)[nH]1] |
| 99 | EMOL_256458365_ 52738565 | 3_Mannose | 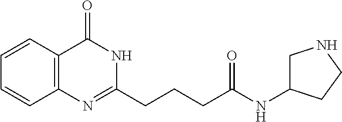 [SMILES: O=C(CCCc1nc2ccccc2c(=O)[nH]1)NC1CCNC1] |
| 100 | EMOL_14823864_ 14823863 | 3_Mannose | 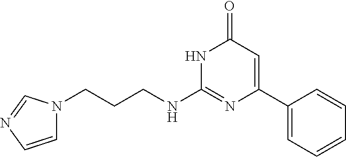 [SMILES: O=c1cc(-c2ccccc2)nc(NCCCn2ccnc2)[nH]1] |
| 101 | EMOL_98440814_ 98440813 | 3_Mannose | 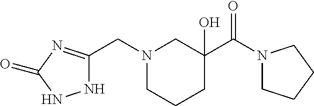 [SMILES: O=C(N1CCCC1)C1(O)CCCN(Cc2nc(=O)[nH][nH]2)C1] |
| 102 | EMOL_256472149_ 71788443 | 3_Mannose | 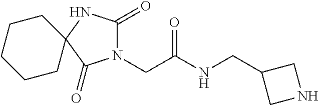 [SMILES: O=C(CN1C(=O)NC2(CCCCC2)C1=O)NCC1CNC1] |
| 103 | EMOL_257994775_ 257994774 | 3_Mannose | 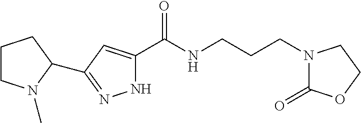 [SMILES: CN1CCCC1c1cc(C(=O)NCCCN2CCOC2=O)[nH]n1] |
| 104 | EMOL_257867559_ 257867558 | 3_Mannose | 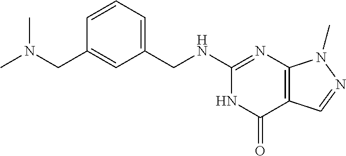 [SMILES: CN(C)Cc1cccc(CNc2nc3c(cnn3C)c(=O)[nH]2)c1] |
| 105 | EMOL_257156079_ 71538964 | 3_Mannose | 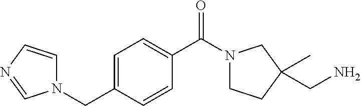 [SMILES: CC1(CN)CCN(C(=O)c2ccc(Cn3ccnc3)cc2)C1] |
| 106 | EMOL_257672757_ 57759101 | 3_Mannose | 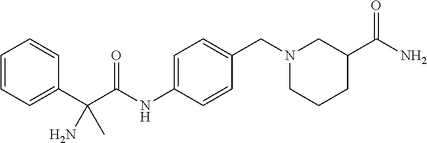 [SMILES: CC(N)(C(=O)Nc1ccc(CN2CCCC(C(N)=O)C2)cc1)c1ccccc1] |
| 107 | EMOL_6820822_ 6820821 | 3_Mannose | 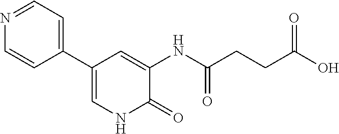 [SMILES: O=C(O)CCC(=O)Nc1cc(-c2ccncc2)c[nH]c1=O] |
| 108 | EMOL_102332549_ 102332548 | 3_Mannose | 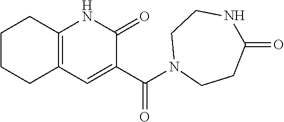 [SMILES: O=C1CCN(C(=O)c2cc3c([nH]c2=O)CCCC3)CCN1] |
| 109 | EMOL_102020236_ 102020235 | 3_Mannose | 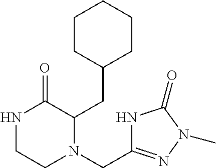 [SMILES: Cn1nc(CN2CCNC(=O)C2CC2CCCCC2)[nH]c1=O] |
| 110 | EMOL_52519632_ 52519631 | 3_Mannose | 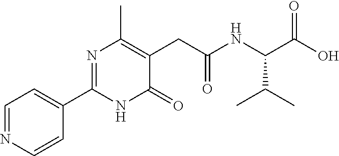 [SMILES: Cc1nc(-c2ccncc2)[nH]c(=O)c1CC(=O)N[C@H](C(=O)O)C(C)C] |
| 111 | EMOL_1438772_ 1438771 | 3_Mannose | 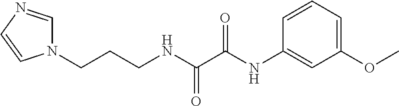 [SMILES: COc1cccc(NC(=O)C(=O)NCCCn2ccnc2)c1] |
| 112 | EMOL_257857514_ 257857513 | 3_Mannose | 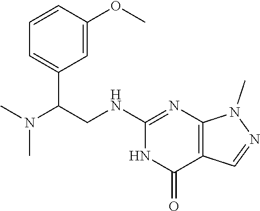 [SMILES: COc1cccc(C(CNc2nc3c(cnn3C)c(=O)[nH]2)N(C)C)c1] |
| 113 | EMOL_53751620_ 53751619 | 3_Mannose | 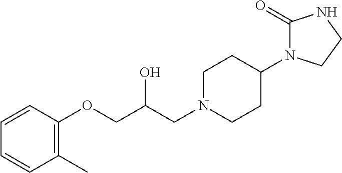 [SMILES: Cc1ccccc1OCC(O)CN1CCC(N2CCNC2=O)CC1] |
| 114 | EMOL_72339494_ 72339493 | 3_Mannose | 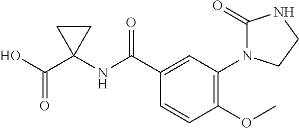 [SMILES: COc1ccc(C(=O)NC2(C(=O)O)CC2)cc1N1CCNC1=O] |
| 115 | EMOL_555132_ 555131 | 3_Mannose | 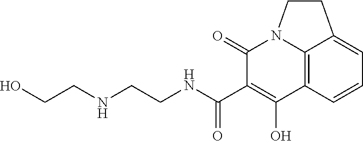 [SMILES: O=C(NCCNCCO)c1c(O)c2cccc3c2n(c1=O)CC3] |
| 116 | EMOL_100896396_ 100896395 | 3_Mannose | 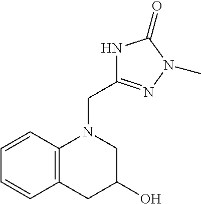 [SMILES: Cn1nc(CN2CC(O)Cc3ccccc32)[nH]c1=O] |
| 117 | EMOL_180483035_ 180483034 | 3_Mannose | 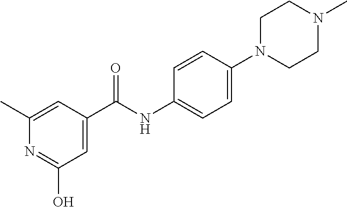 |
| 118 | EMOL_257725483_ 57553880 | 3_Mannose | 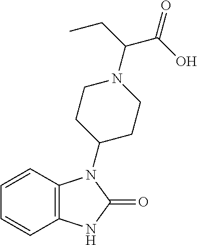 [SMILES: CCC(C(=O)O)N1CCC(n2c(=O)[nH]c3ccccc32)CC1] |
| 119 | EMOL_257464685_ 71086166 | 3_Mannose | 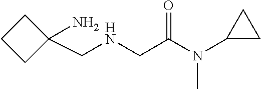 |
| 120 | EMOL_31905486_ 31905485 | 3_Mannose | 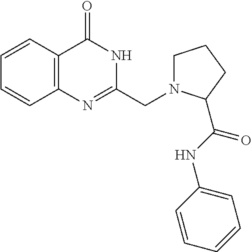 [SMILES: O=C(Nc1ccccc1)C1CCCN1Cc1nc2ccccc2c(=O)[nH]1] |
| 121 | EMOL_681596_ 681595 | 3_Mannose | 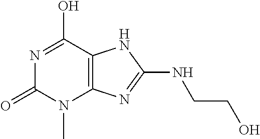 [SMILES: Cn1c(=O)nc(O)c2[nH]c(NCCO)nc21] |
| 122 | EMOL_57259884_ 57259883 | 3_Mannose | 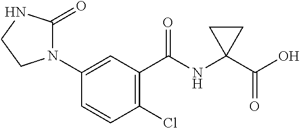 [SMILES: O=C(NC1(C(=O)O)CC1)c1cc(N2CCNC2=O)ccc1Cl] |
| 123 | EMOL_257680686_ 71824453 | 3_Mannose | 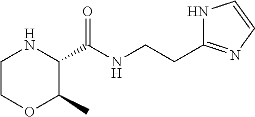 [SMILES: C[C@H]1OCCN[C@@H]1C(=O)NCCc1ncc[nH]1] |
| 124 | EMOL_100108686_ 100108685 | 3_Mannose | 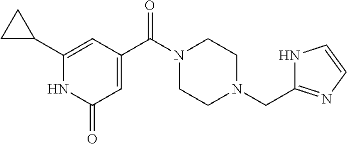 [SMILES: O=C(c1cc(C2CC2)[nH]c(=O)c1)N1CCN(Cc2ncc[nH]2)CC1] |
| 125 | EMOL_42873641_ 42873640 | 3_Mannose | 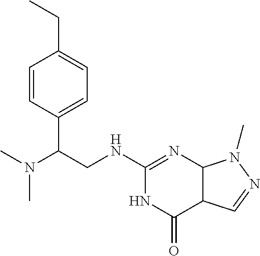 [SMILES: CCc1ccc(C(CNC2=NC3C(C=NN3C)C(=O)N2)N(C)C)cc1] |
| 126 | EMOL_70095256_ 70095255 | 3_Mannose | 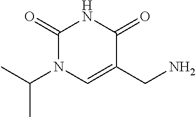 |
| 127 | EMOL_32797522_ 32797521 | 3_Mannose | 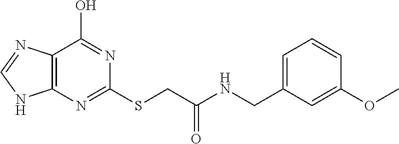 [SMILES: COc1cccc(CNC(=O)CSc2nc(O)c3nc[nH]c3n2)c1] |
| 128 | EMOL_257686560_ 71546857 | 3_Mannose | 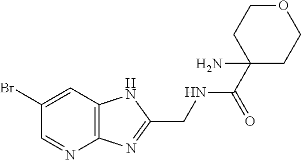 [SMILES: NC1(C(=O)NCc2nc3ncc(Br)cc3[nH]2)CCOCC1] |
| 129 | EMOL_55630570_ 55630569 | 3_Mannose | 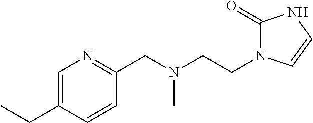 [SMILES: CCc1ccc(CN(C)CCn2cc[nH]c2=O)nc1] |
| 130 | EMOL_37734822_ 37734821 | 3_Mannose | 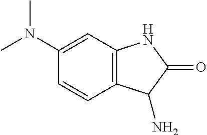 |
| 131 | EMOL_55704932_ 55704931 | 3_Mannose | 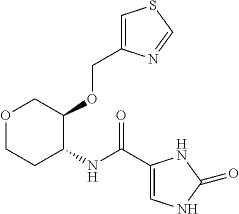 [SMILES: O=C(N[C@@H]1CCOC[C@H]1OCc1cscn1)c1c[nH]c(=O)[nH]1] |
| 132 | EMOL_5559462_ 5559461 | 3_Mannose | 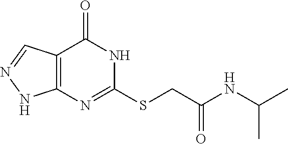 [SMILES: CC(C)NC(=O)CSc1nc2[nH]ncc2c(=O)[nH]1] |
| 133 | EMOL_72081290_ 72081289 | 3_Mannose | 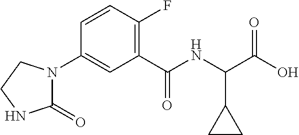 [SMILES: O=C(NC(C(=O)O)C1CC1)c1cc(N2CCNC2=O)ccc1F] |
| 134 | EMOL_72963349_ 72963348 | 3_Mannose | 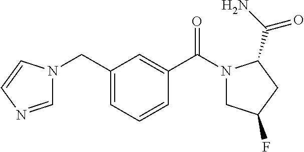 [SMILES: NC(=O)[C@@H]1C[C@@H](F)CN1C(=O)c1cccc(Cn2ccnc2)c1] |
| 135 | EMOL_256706354_ 57129468 | 3_Mannose | 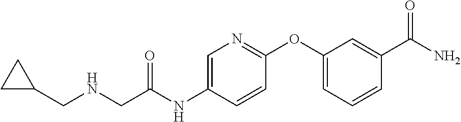 [SMILES: NC(=O)c1cccc(Oc2ccc(NC(=O)CNCC3CC3)cn2)c1] |
| 136 | EMOL_4750938_ 4750937 | 3_Mannose | 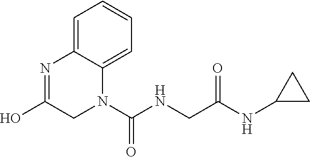 [SMILES: O=C(CNC(=O)N1CC(O)=Nc2ccccc21)NC1CC1] |
| 137 | EMOL_11474652_ 11474651 | 3_Mannose | 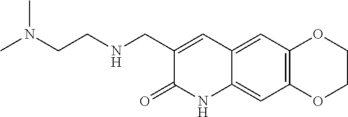 [SMILES: CN(C)CCNCc1cc2cc3c(cc2[nH]c1=O)OCCO3] |
| 138 | EMOL_72093374_ 72093373 | 3_Mannose | 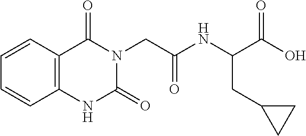 [SMILES: O=C(Cn1c(=O)[nH]c2ccccc2c1=O)NC(CC1CC1)C(=O)O] |
| 139 | EMOL_11474796_ 11474795 | 3_Mannose | 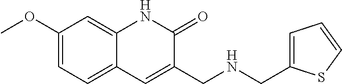 [SMILES: COc1ccc2cc(CNCc3cccs3)c(=O)[nH]c2c1] |
| 140 | EMOL_232854551_ 232854550 | 3_Mannose | 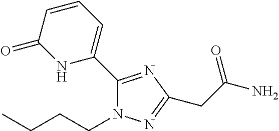 [SMILES: CCCCn1nc(CC(N)=O)nc1-c1cccc(=O)[nH]1] |
| 141 | EMOL_55694051_ 55694050 | 3_Mannose | 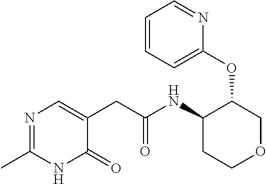 [SMILES: Cc1ncc(CC(=O)N[C@@H]2CCOC[C@H]2Oc2ccccn2)c(=O)[nH]1] |
| 142 | EMOL_20410050_ 20410049 | 3_Mannose | 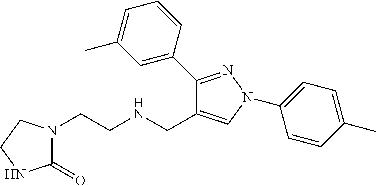 [SMILES: Cc1ccc(-n2cc(CNCCN3CCNC3=O)c(-c3cccc(C)c3)n2)cc1] |
| 143 | EMOL_27079492_ 27079491 | 3_Mannose | 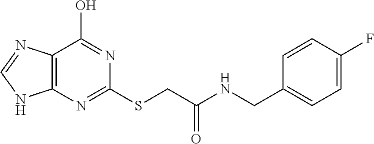 [SMILES: O=C(CSc1nc(O)c2nc[nH]c2n1)NCc1ccc(F)cc1] |
| 144 | EMOL_32331720_ 32331719 | 3_Mannose | 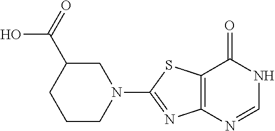 [SMILES: O=C(O)C1CCCN(c2nc3nc[nH]c(=O)c3s2)C1] |
| 145 | EMOL_558555_ 558554 | 3_Mannose | 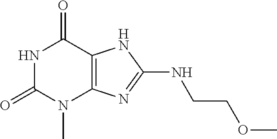 [SMILES: COCCNc1nc2c([nH]1)c(=O)[nH]c(=O)n2C] |
| 146 | EMOL_11474508_ 11474507 | 3_Mannose | 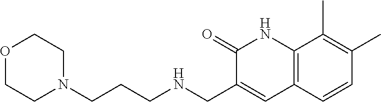 [SMILES: Cc1ccc2cc(CNCCCN3CCOCC3)c(=O)[nH]c2c1C] |
| 147 | EMOL_256898707_ 52578280 | 3_Mannose | 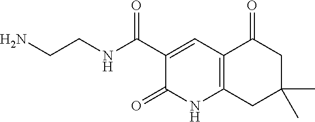 [SMILES: CC1(C)CC(=O)c2cc(C(=O)NCCN)c(=O)[nH]c2C1] |
| 148 | EMOL_76028376_ 76028375 | 3_Mannose | 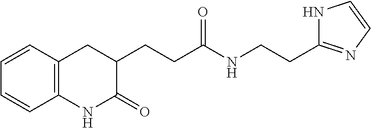 [SMILES: O=C(CCC1Cc2ccccc2NC1=O)NCCc1ncc[nH]1] |
| 149 | EMOL_3643564_ 3643563 | 3_Mannose | 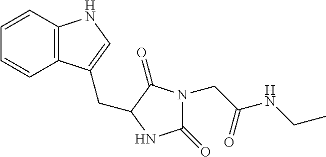 [SMILES: CCNC(=O)CN1C(=O)NC(Cc2c[nH]c3ccccc23)C1=O] |
| 150 | EMOL_72341788_ 72341787 | 3_Mannose | 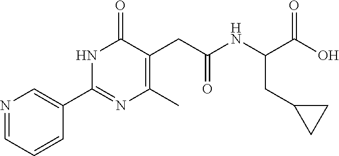 [SMILES: Cc1nc(-c2cccnc2)[nH]c(=O)c1CC(=O)NC(CC1CC1)C(=O)O] |
| 151 | EMOL_52524290_ 52524289 | 3_Mannose | 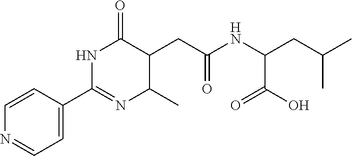 [SMILES: CC(C)CC(NC(=O)CC1C(=O)NC(c2ccncc2)=NC1C)C(=O)O] |
| 152 | EMOL_257441568_ 71490402 | 3_Mannose | 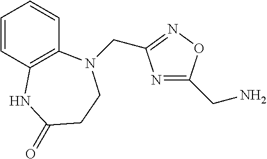 [SMILES: NCc1nc(CN2CCC(=O)Nc3ccccc32)no1] |
| 153 | EMOL_5630576_ 5630575 | 3_Mannose | 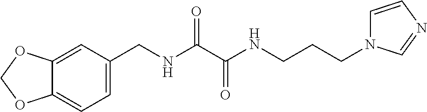 [SMILES: O=C(NCCCn1ccnc1)C(=O)NCc1ccc2c(c1)OCO2] |
| 154 | EMOL_32035120_ 32035119 | 3_Mannose | 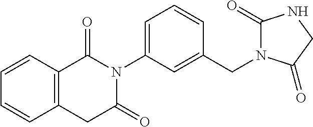 [SMILES: O=C1CNC(=O)N1Cc1cccc(N2C(=O)Cc3ccccc3C2=O)c1] |
| 155 | EMOL_232864752_ 232864751 | 3_Mannose | 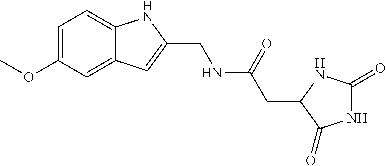 [SMILES: COc1ccc2[nH]c(CNC(=O)CC3NC(=O)NC3=O)cc2c1] |
| 156 | EMOL_4975385_ 4975384 | 3_Mannose | 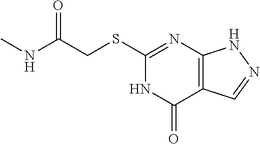 [SMILES: CNC(=O)CSc1nc2[nH]ncc2c(=O)[nH]1] |
| 157 | EMOL_36326756_ 36326755 | 3_Mannose | 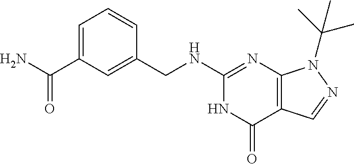 [SMILES: CC(C)(C)n1ncc2c(=O)[nH]c(NCc3cccc(C(N)=O)c3)nc21] |
| 158 | EMOL_43133889_ 43133888 | 3_Mannose | 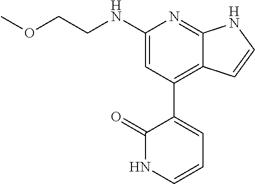 [SMILES: COCCNc1cc(-c2ccc[nH]c2=O)c2cc[nH]c2n1] |
| 159 | EMOL_4975297_ 4975296 | 3_Mannose | 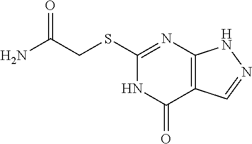 [SMILES: NC(=O)CSc1nc2[nH]ncc2c(=O)[nH]1] |
| 160 | EMOL_32385294_ 32385293 | 3_Mannose | 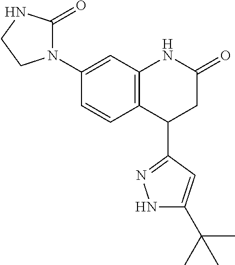 [SMILES: CC(C)(C)c1cc(C2CC(=O)Nc3cc(N4CCNC4=O)ccc32)n[nH]1] |
| 161 | EMOL_72807921_ 72807920 | 3_Mannose | 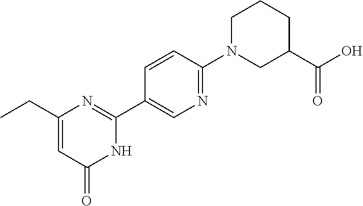 [SMILES: CCc1cc(=O)[nH]c(-c2ccc(N3CCCC(C(=O)O)C3)nc2)n1] |
| 162 | EMOL_257548186_ 53628721 | 3_Mannose | 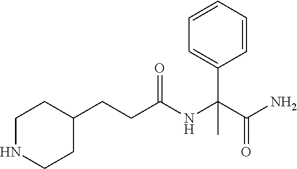 [SMILES: CC(NC(=O)CCC1CCNCC1)(C(N)=O)c1ccccc1] |
| 163 | EMOL_257013437_ 257013436 | 3_Mannose | 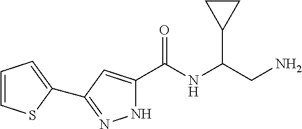 [SMILES: NCC(NC(=O)c1cc(-c2cccs2)n[nH]1)C1CC1] |
| 164 | EMOL_32257987_ 32257986 | 3_Mannose | 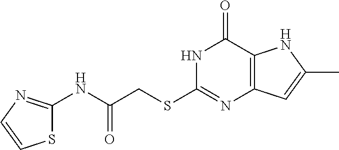 [SMILES: Cc1cc2nc(SCC(=O)Nc3nccs3)[nH]c(=O)c2[nH]1] |
| 165 | EMOL_4718149_ 4718148 | 3_Mannose | 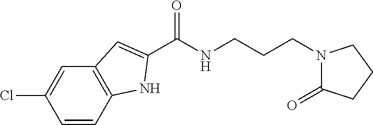 [SMILES: O=C(NCCCN1CCCC1=O)c1cc2cc(Cl)ccc2[nH]1] |
| 166 | EMOL_257987864_ 257987863 | 3_Mannose | 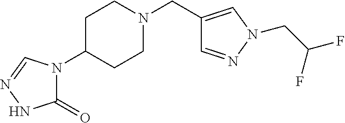 [SMILES: O=c1[nH]ncn1C1CCN(Cc2cnn(CC(F)F)c2)CC1] |
| 167 | EMOL_256984435_ 256984434 | 3_Mannose | 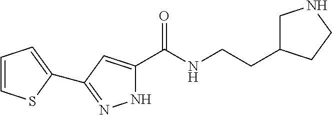 [SMILES: O=C(NCCC1CCNC1)c1cc(-c2cccs2)n[nH]1] |
| 168 | EMOL_43164496_ 43164495 | 3_Mannose | 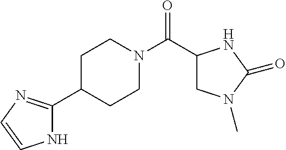 [SMILES: CN1CC(C(=O)N2CCC(c3ncc[nH]3)CC2)NC1=O] |
| 169 | EMOL_27508985_ 27508984 | 3_Mannose | 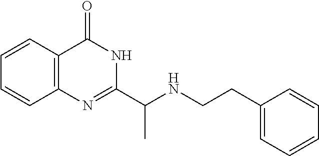 [SMILES: CC(NCCc1ccccc1)c1nc2ccccc2c(=O)[nH]1] |
| 170 | EMOL_110914416_ 110914415 | 3_Mannose | 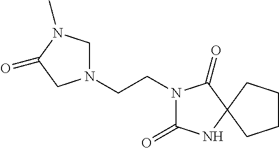 [SMILES: CN1CN(CCN2C(=O)NC3(CCCC3)C2=O)CC1=O] |
| 171 | EMOL_101756206_ 101756205 | 3_Mannose | 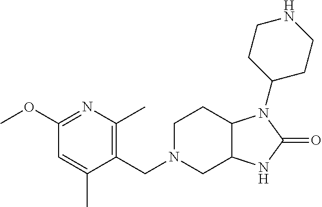 [SMILES: COc1cc(C)c(CN2CCC3C(C2)NC(=O)N3C2CCNCC2)c(C)n1] |
| 172 | EMOL_100522890_ 100522889 | 3_Mannose | 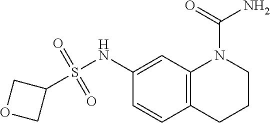 [SMILES: NC(=O)N1CCCc2ccc(NS(=O)(=O)C3COC3)cc21] |
| 173 | EMOL_101899777_ 101899776 | 3_Mannose | 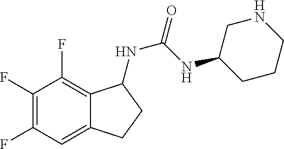 [SMILES: O=C(NC1CCc2cc(F)c(F)c(F)c21)N[C@@H]1CCCNC1] |
| 174 | EMOL_99342595_ 99342594 | 3_Mannose | 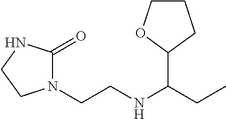 [SMILES: CCC(NCCN1CCNC1=O)C1CCCO1] |
| 175 | EMOL_111726824_ 111726823 | 3_Mannose | 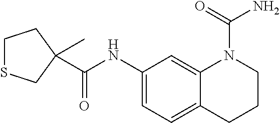 [SMILES: CC1(C(=O)Nc2ccc3c(c2)N(C(N)=O)CCC3)CCSC1] |
| 176 | EMOL_110793024_ 110793023 | 3_Mannose | 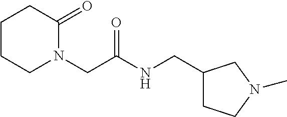 [SMILES: CN1CCC(CNC(=O)CN2CCCCC2=O)C1] |
| 177 | EMOL_98508770_ 98508769 | 3_Mannose | 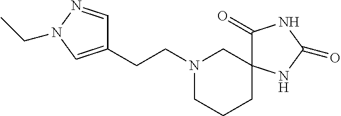 [SMILES: CCn1cc(CCN2CCCC3(C2)NC(=O)NC3=O)cn1] |
| 178 | EMOL_98145595_ 98145594 | 3_Mannose | 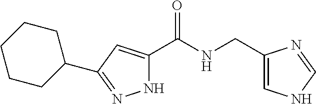 [SMILES: O=C(NCc1c[nH]cn1)c1cc(C2CCCCC2)n[nH]1] |
| 179 | EMOL_101148344_ 101148343 | 3_Mannose | 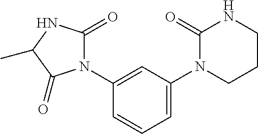 [SMILES: CC1NC(=O)N(c2cccc(N3CCCNC3=O)c2)C1=O] |
| 180 | EMOL_110764903_ 110764902 | 3_Mannose | 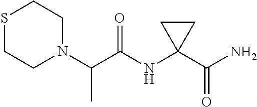 [SMILES: CC(C(=O)NC1(C(N)=O)CC1)N1CCSCC1] |
| 181 | EMOL_257563502_ 52417048 | 3_Mannose | 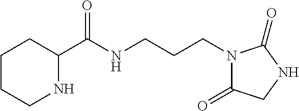 [SMILES: O=C(NCCCN1C(=O)CNC1=O)C1CCCCN1] |
| 182 | EMOL_111017186_ 111017185 | 3_Mannose | 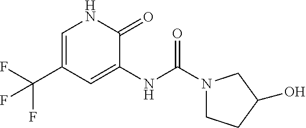 [SMILES: O=C(Nc1cc(C(F)(F)F)c[nH]c1=O)N1CCC(O)C1] |
| 183 | EMOL_111453491_ 111453490 | 3_Mannose | 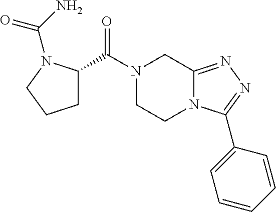 [SMILES: NC(=O)N1CCC[C@H]1C(=O)N1CCn2c(nnc2-c2ccccc2)C1] |
| 184 | EMOL_111709519_ 111709518 | 3_Mannose | 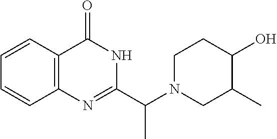 [SMILES: CC1CN(C(C)c2nc3ccccc3c(=O)[nH]2)CCC1O] |
| 185 | EMOL_102203374_ 102203373 | 3_Mannose | 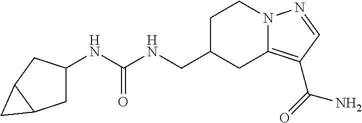 [SMILES: NC(=O)c1cnn2c1CC(CNC(=O)NC1CC3CC3C1)CC2] |
| 186 | EMOL_111330639_ 111330638 | 3_Mannose | 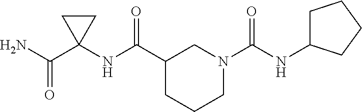 [SMILES: NC(=O)C1(NC(=O)C2CCCN(C(=O)NC3CCCC3)C2)CC1] |
| 187 | EMOL_110944932_ 110944931 | 3_Mannose | 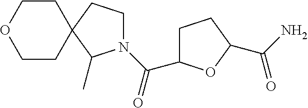 [SMILES: CC1N(C(=O)C2CCC(C(N)=O)O2)CCC12CCOCC2] |
| 188 | EMOL_257631739_ 71148726 | 3_Mannose (Adjacent) | 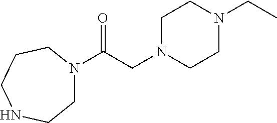 [SMILES: CCN1CCN(CC(=O)N2CCCNCC2)CC1] |
| 189 | EMOL_257691209_ 71230189 | 3_Mannose (Adjacent) | 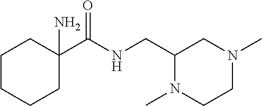 [SMILES: CN1CCN(C)C(CNC(=O)C2(N)CCCCC2)C1] |
| 190 | EMOL_102888595_ 102888594 | 3_Mannose (Adjacent) | 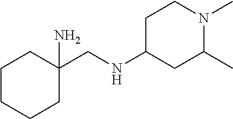 [SMILES: CC1CC(NCC2(N)CCCCC2)CCN1C] |
| 191 | EMOL_102862622_ 102862621 | 3_Mannose (Adjacent) | 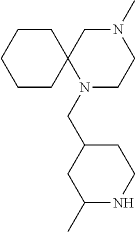 [SMILES: CC1CC(CN2CCN(C)CC23CCCCC3)CCN1] |
| 192 | EMOL_102700041_ 102700040 | 3_Mannose (Adjacent) | 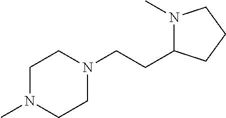 |
| 193 | EMOL_257707164_ 71193084 | 3_Mannose (Adjacent) | 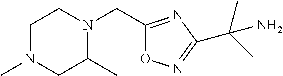 [SMILES: CC1CN(C)CCN1Cc1nc(C(C)(C)N)no1] |
| 194 | EMOL_102341427_ 102341426 | 3_Mannose (Adjacent) | 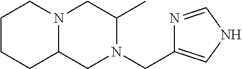 [SMILES: CC1CN2CCCCC2CN1Cc1c[nH]cn1] |
| 195 | EMOL_257703645_ 71212245 | 3_Mannose (Adjacent) | 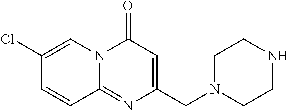 [SMILES: O=c1cc(CN2CCNCC2)nc2ccc(Cl)cn12] |
| 196 | EMOL_101763856_ 101763855 | 3_Mannose (Adjacent) | 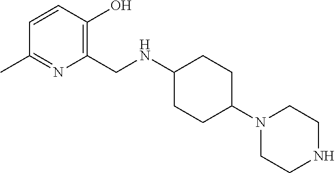 [SMILES: Cc1ccc(O)c(CNC2CCC(N3CCNCC3)CC2)n1] |
| 197 | EMOL_102781038_ 102781037 | 3_Mannose (Adjacent) | 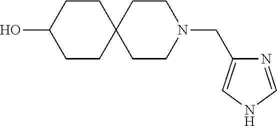 [SMILES: OC1CCC2(CC1)CCN(Cc1c[nH]cn1)CC2] |
| 198 | EMOL_102781026_ 102781025 | 3_Mannose (Adjacent) | 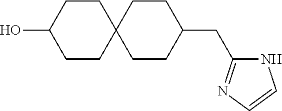 [SMILES: OC1CCC2(CC1)CCC(Cc1ncc[nH]1)CC2] |
| 199 | EMOL_71271960_ 71271959 | 3_Mannose (Adjacent) | 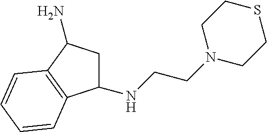 [SMILES: NC1CC(NCCN2CCSCC2)c2ccccc21] |

In some embodiments, a targeting agent is capable of binding to CD204. In some embodiments, a targeting agent is a CD204 ligand or portion thereof. In some embodiments, the targeting agent comprises at least a portion of lipid A, oxidized low-density lipoprotein (LDL), acetylated LDL, malondialdehyde modified LDL, maleylated LDL, lysophasphatidylcholine, phophatidic acid, cholesterol, Apo A-I, Apo E, glycated type IV collagen, modified collagen type I, III and IV, biglycan, decorin, albumin, advanced glycation end product bovine serum albumin, b-amyloid fibrils, calreticulin, gp96, an HSP70 protein, a lipopolysaccharide, lymphotoxin-alpha, CpG DNA, calciprotein particles, a Neisseria meningitides surface protein, C reactive protein, hepatitis C virus NS3 protein, or Tamm-Horsfall protein. A targeting agent capable of binding to CD204 is able to target a cell of a macrophage network (e.g., a tumor associated macrophage) by binding to CD204 present on the surface of a cell.

In some embodiments, a targeting agent is capable of binding to CD163. In some embodiments, a targeting agent is a CD163 ligand or portion thereof. In some embodiments, the targeting agent comprises at least a portion of haptoglobin, hemoglobin, tumor necrosis factor-α (TNF-α)-like weak inducer of the apoptosis (TWEAK), gram positive and gram negative bacteria, or casein kinase II subunit beta.

### Imaging Agents

In certain aspects, the disclosed methods comprise the use of an imaging agent. In some embodiments, an imaging agent is directed to a macrophage network, for example, using a targeting agent. In some embodiments, an imaging agent is capable of imaging a macrophage network. In some embodiments, an imaging agent is 5-carboxyfluorescein, fluorescein-5-isothiocyanate, fluorescein-6-isothiocyanate, 6-carboxyfluorescein, tetramethylrhodamine-6-isothiocyanate, 5-carboxytetramethylrhodamine, 5-carboxy rhodol derivatives, tetramethyl and tetraethyl rhodamine, diphenyldimethyl and diphenyldiethyl rhodamine, dinaphthyl rhodamine, rhodamine 101 sulfonyl chloride, Cy3, Cy3B, Cy3.5, Cy5, Cy5.5, Cy7, DyLight650, IRDye650, IRDye680, DyLight750, Alexa Fluor 647, Alexa Fluor 750, IR800CW, ICG, Green Fluorescent Protein, EBFP, EBFP2, Azurite, mKalamal, ECFP, Cerulean, CyPet, YFP, Citrine, Venus, YPet, a gadolinium chelate, an iron oxide particle, a super paramagnetic iron oxide particle, an ultra-small paramagnetic particle, a manganese chelate, gallium containing agent, ⁶⁴Cu diacetyl-bis(N⁴-methylthiosemicarbazone), ¹⁸F-fluorodeoxyglucose, ¹⁸F-fluoride, 3′-deoxy-3′-[¹⁸F]fluorothymidine, ¹⁸F-fluoromisonidazole, technetium-99m, thallium, iodine, barium-sulphate, or a combination thereof. In some embodiments, an imaging agent is conjugated to one or more additional agents, such as a targeting agent, a cytotoxic agent, or a macrophage polarizing agent.

In some embodiments, an imaging agent is used to visualize a macrophage network. Visualization of a macrophage network is useful in, for example, visualizing a tumor in a subject (e.g., determining the size and/or the location of the tumor in the subject). In some embodiments, an imaging agent is directed to a macrophage network by a targeting agent and the imaging agent is detected, thereby visualizing a tumor in a subject. In some embodiments, an imaging agent is conjugated to a cytotoxic agent and directed to a macrophage network by a targeting agent, thereby visualizing a tumor and also disrupting the macrophage network. In some embodiments, an imaging agent is directed to a macrophage network, thereby visualizing a tumor, and a cytotoxic agent is separately directed to the macrophage network, thereby disrupting the macrophage network.

### Therapeutic and Cytotoxic Agents

Described herein, in some aspects, are methods using compounds comprising a therapeutic and/or cytotoxic agent. In some embodiments, a cytotoxic agent is used to treat or ameliorate cancer. In some embodiments, a cytotoxic agent is a chemotherapeutic agent, an anti-tubulin agent, a DNA modifying agent, or a small interfering ribonucleic acid. In some embodiments, a cytotoxic agent is auristatin, a dolastatin, auristatin E, Monomethyl auristatin E (MMAE), Monomethyl auristatin F (MMAF), dimethylvaline-valine-dolaisoleuine-dolaproine-phenylalanine-p-phenylenediamine (AFP), 5-benzoylvaleric acid-auristatin E ester (AEVB), auristatin EB (AEB), Ansamitocin, Mertansine/emtansine (DM1), ravtansine/soravtansine (DM4), Duocamycin, Calicheamicin, and pyrrolobenzodiazepines. In some embodiments, a cytotoxic agent is used for killing one or more cancer cells. In some embodiments, a cytotoxic agent is used for killing one or more cells of a macrophage network (e.g., one or more tumor associated macrophages).

In some embodiments, a therapeutic agent is used to treat or ameliorate a granulomatous disease (e.g., tuberculosis). In some embodiments, a therapeutic agent is an antibacterial agent. In some embodiments, an antibacterial agent is an antibiotic. In some embodiments, an antibiotic is penicillin, streptomycin, actinomycin D, ampicillin, blasticidin, carbenicillin, cefotaxime, fosmidomycin, gentamicin, kanamycin, neomycin, polymyxin B, isoniazid, rifampin, ethambutol, purazinamide, amikacin, kanamycin, capreomycin or any combination thereof. In some embodiments, a therapeutic agent is an anti-inflammatory agent. In some embodiments, an anti-inflammatory agent is a nonsteroidal anti-inflammatory drug (NSAID), a glucocorticoid, or a disease-modifying agent of rheumatoid diseases (DMARD). In some embodiments, an NSAID is asprin, celecoxib, diclofenac, diflunisal, etodolac, ibuprofen, indomethacin, ketoprofin, ketorolac, nabumetone, naproxen, oxaprozin, piroxicam, salsalate, sulindac, or tolmetin. In some embodiments, a glucocorticoid is beclomethasone, betamethasone, budesonide, cortisone, dexamethasone, hydrocortisone, methylprednisolone, prednisolone, prednisone, or triamcinolone. In some embodiments, a DMARD is methotrexate, sulfasalazine, hydroxychloroquinine, leflunomide, azathioprine, cyclosporine, etanercept, adalimumab, infliximab, certolizumab pegol, or golimumab.

### Macrophage Polarization

In some embodiments, a macrophage network is disrupted by altering the polarization of a macrophage. Without wishing to be bound by any particular theory, macrophages are thought to be polarized in response to stimuli. Examples of polarized macrophages include, for example, M1 macrophages, M2 macrophages, and tumor associated macrophages. In some embodiments, a compound of the present disclosure comprises a macrophage polarizing agent. In some embodiments, a macrophage polarizing agent is directed to a macrophage of a macrophage network, thereby altering the polarization of the macrophage and disrupting the macrophage network. For example, a macrophage polarizing agent is provided to a macrophage network comprising tumor associated macrophages, thereby polarizing the tumor associated macrophages and disrupting the macrophage network. In some embodiments, a macrophage polarizing agent is CpG DNA.

### Pharmaceutical Compositions

Compounds described herein can be formulated as a pharmaceutical composition for administration purposes. Pharmaceutical compositions may be formulated in a conventional manner using one or more physiologically acceptable carriers including excipients and auxiliaries which facilitate processing of the active compounds into preparations which can be used pharmaceutically. Proper formulation is dependent upon the route of administration chosen. Any techniques, carriers, and excipients may be used as suitable and as understood in the art. A summary of pharmaceutical compositions which may be used herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins; 1999), herein incorporated by reference in their entirety.

In certain embodiments, compositions comprise a compound as disclosed herein and a pharmaceutically acceptable diluent(s), excipient(s), or carrier(s). In addition, the compounds described herein can be administered as pharmaceutical compositions in which the compounds are mixed with other active ingredients, as in combination therapy. In some embodiments, the pharmaceutical compositions may include other medicinal or pharmaceutical agents, carriers, adjuvants, such as preserving, stabilizing, wetting or emulsifying agents, solution promoters, salts for regulating the osmotic pressure, and/or buffers. In addition, the pharmaceutical compositions can also contain other therapeutically valuable substances.

A pharmaceutical composition, as used herein, refers to a mixture of compounds described herein with other chemical components, such as carriers, stabilizers, diluents, dispersing agents, suspending agents, thickening agents, and/or excipients. The pharmaceutical composition facilitates administration of the compound to an organism. In practicing the methods of treatment or use provided herein, therapeutically effective amounts of one or more compounds described herein are administered in a pharmaceutical composition to a mammal having a disease, disorder, or condition to be treated (e.g., cancer or a granulomatous disease). In some embodiments, the mammal is a human. A therapeutically effective amount can vary widely depending on the severity of the disease, the age and relative health of the subject, the potency of the compound used and other factors. The compounds can be used singly or in combination with one or more therapeutic agents as components of mixtures.

### Injectable Formulations

Formulations suitable for intramuscular, subcutaneous, or intravenous injection may include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles include water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. Formulations suitable for subcutaneous injection may also contain additives such as preserving, wetting, emulsifying, and dispensing agents. Prevention of the growth of microorganisms can be ensured by various antibacterial and antifungal agents, such as parabens, chlorobutanol, phenol, sorbic acid, and the like. It may also be desirable to include isotonic agents, such as sugars, sodium chloride, and the like. Prolonged absorption of the injectable pharmaceutical form can be brought about by the use of agents delaying absorption, such as aluminum monostearate and gelatin.

For intravenous injections, compounds described herein may be formulated in aqueous solutions, in physiologically compatible buffers such as Hank's solution, Ringer's solution, physiological saline buffer, or other suitable solutions. For transmucosal administration, penetrants appropriate to the barrier to be permeated are used in the formulation. For other parenteral injections, appropriate formulations may include aqueous or nonaqueous solutions, preferably with physiologically compatible buffers or excipients.

Parenteral injections may involve bolus injection or continuous infusion. Formulations for injection may be presented in unit dosage form, e.g., in ampoules or in multi dose containers, with an added preservative. The pharmaceutical composition described herein may be in a form suitable for parenteral injection as a sterile suspensions, solutions or emulsions in oily or aqueous vehicles, and may contain formulatory agents such as suspending, stabilizing and/or dispersing agents. Pharmaceutical formulations for parenteral administration include aqueous solutions of the active compounds in water soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the active ingredient may be in powder form for constitution with a suitable vehicle, e.g., sterile pyrogen-free water, before use.

### Solid Oral Dosage Formulations

The pharmaceutical solid dosage forms comprise a compound described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof.

The pharmaceutical solid dosage forms compounds described herein can be formulated to provide a controlled release of the composition. Controlled release refers to the release of the composition from a dosage form in which it is incorporated according to a desired profile over an extended period of time. Controlled release profiles include, for example, sustained release, prolonged release, pulsatile release, and delayed release profiles. In contrast to immediate release compositions, controlled release compositions allow delivery of an agent to a subject over an extended period of time according to a predetermined profile. Such release rates can provide therapeutically effective levels of agent for an extended period of time and thereby provide a longer period of pharmacologic response while minimizing side effects as compared to conventional rapid release dosage forms. Such longer periods of response provide for many inherent benefits that are not achieved with the corresponding short acting, immediate release preparations.

In some embodiments, the solid dosage forms described herein can be formulated as enteric coated delayed release oral dosage forms, i.e., as an oral dosage form of a pharmaceutical composition as described herein which utilizes an enteric coating to affect release in the small intestine of the gastrointestinal tract. The enteric coated dosage form may be a compressed or molded or extruded tablet/mold (coated or uncoated) containing granules, powder, pellets, beads or particles of the active ingredient and/or other composition components, which are themselves coated or uncoated. The enteric coated oral dosage form may also be a capsule (coated or uncoated) containing pellets, beads or granules of the solid carrier or the composition, which are themselves coated or uncoated.

In other embodiments, the formulations described herein are delivered using a pulsatile dosage form. A pulsatile dosage form is capable of providing one or more immediate release pulses at predetermined time points after a controlled lag time or at specific sites.

In some embodiments, pharmaceutical formulations are provided that include particles of the compositions described herein and at least one dispersing agent or suspending agent for administration to a subject. The formulations may be a powder and/or granules for suspension, and upon admixture with water, a substantially uniform suspension is obtained.

In addition to the additives listed above, the liquid formulations can also include inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, sodium lauryl sulfate, sodium doccusate, cholesterol, cholesterol esters, taurocholic acid, phosphotidylcholine, oils, such as cottonseed oil, groundnut oil, corn germ oil, olive oil, castor oil, and sesame oil, glycerol, tetrahydrofurfuryl alcohol, polyethylene glycols, fatty acid esters of sorbitan, or mixtures of these substances, and the like.

### EXAMPLES

### Example 1: Visualization of a Macrophage Network Surrounding a Tumor

Athymic mice were stereotactically implanted with 400,000 U87 human glioma cells in the right striatum of the brain. Twelve days later, mice were intravenously injected (tail vein) with 200 μl of a 10 mg/ml dextran-tetramethylrhodamine-saline solution (3,000 MW or 10,000 MW Anionic, Lysine Fixable, Life Technologies) and dextran was allowed to circulate for 2 minutes before mice were euthanized and brain tumors harvested. Tumors were fixed overnight at 4° C. in 4% PFA in PBS, followed by sinking in graded sucrose solution (15% to 30% in PBS). Tumors were mounted in optimal cutting temperature (OCT) compound and sectioned on cryostat at 60 micron thickness. Immunofluorescent staining was performed as described (Barnett F H et al., Macrophages form functional vascular mimicry channels in vivo. Scientific reports. 2016; 6:36659) using manufacturer's recommended antibody dilutions. All images were gathered with a confocal laser-scanning microscope (LSM 700 or 710, Carl Zeiss) utilizing a Plan-Apochromat 20×/0.8, Plan-Apochromat 63×/1.4 Oil DIC, C-Apochromat 40×/1.2W Korr UV-VIS objective lens (Carl Zeiss) and processed with the ZEN 2010 software (Carl Zeiss). Scanning was performed in sequential laser emission mode to avoid scanning at other wavelengths. Three-dimensional reconstructions were generated using ZEN 2010. Z-stacks were acquired using a Zeiss 710 laser scanning confocal microscope using a 20× objective (1 μm step size), or a 63× objective (0.3 μm step size) and assembled in the Zen software (4 experiments n=3-5 per experiment).

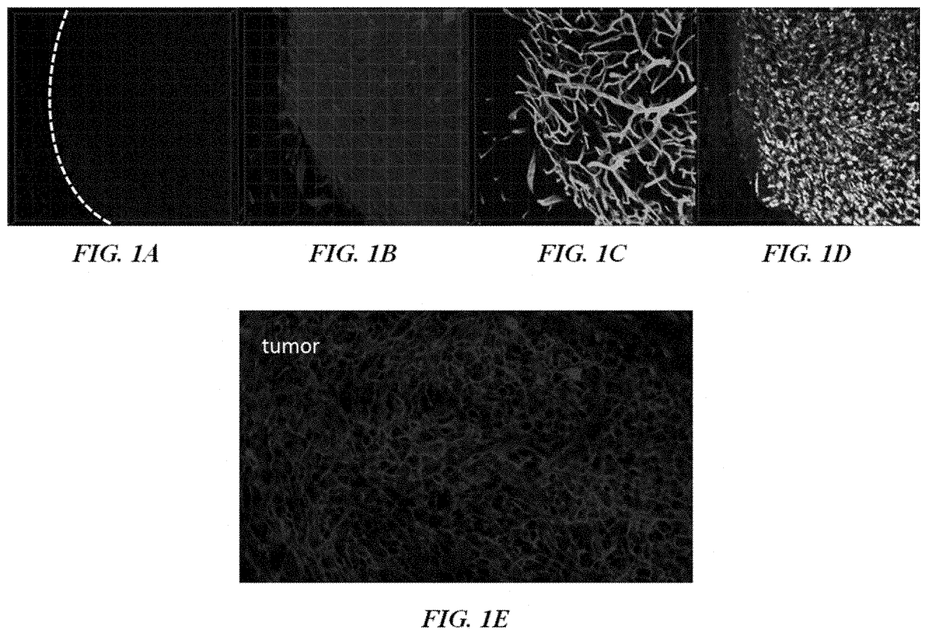

Results are shown in FIGS. 1A-E. FIG. 1A shows imaging of a tumor in the blue fluorescent channel using Hoechst nuclear stain, defining the edge of the tumor as shown (white dashed line). FIG. 1B shows imaging of the tumor in FIG. 1A in the red fluorescent channel using the 3,000 MW red dextran dye, demonstrating the penetration of the dextran dye into the tumor via a macrophage network. FIG. 1C shows imaging of the tumor in FIG. 1A in the green fluorescent channel using fluorescent antibodies specific for the blood vessel marker CD31, showing the blood vessels both outside the tumor (left side) as well as within the tumor. FIG. 1D shows imaging of the tumor in FIG. 1A in the far red fluorescent channel (depicted as gold) using fluorescent antibodies specific for the macrophage marker Iba1, demonstrating the presence of the macrophage network within the tumor. FIG. 1E shows imaging of a tumor from another mouse, injected with 10,000 MW red dextran dye, demonstrating the penetration of 10,000 MW dye into the tumor via a macrophage network.

### Example 2: Disrupting a Macrophage Network in a Subject

An individual with cancer is identified. A compound comprising an imaging agent and a CD206 targeting agent is provided to the individual, thereby visualizing the macrophage network around the tumor. Next, a compound comprising a CD206 targeting agent and a cytotoxic agent is provided to the individual. Following this, the compound comprising the imaging agent is again provided to the individual. Observation of a reduction in the size and complexity of the macrophage network indicates that the network has been disrupted.

### Example 3: Targeting Tumor Associated Macrophages to Treat Cancer

An individual with cancer is identified. A size of a tumor in the individual is measured by magnetic resonance imaging (MRI) with gadolinium contrast dye. A compound comprising a CD206 targeting agent and a cytotoxic agent is provided to the individual. Following treatment, the size of the tumor is measuring using MRI to determine if the tumor size has been reduced.

### Example 4: Targeting Tumor Associated Macrophages to Treat Tuberculosis

An individual with tuberculosis is identified. A compound comprising a CD206 targeting agent and an antibiotic is provided to the individual. Following treatment, a chest X-ray is performed on the individual to determine if the degree of tuberculosis infection has been reduced or eliminated.

### Example 5: Combination Therapy Using a Targeting Agent and a CAR T-Cell Therapy to Treat Cancer

An individual with cancer is identified. A size of a tumor in the individual is measured by MRI with gadolinium contrast dye. First, a compound comprising a CD206 targeting agent and a cytotoxic agent is provided to the individual. Next, a CAR T-cell therapy is provided to the patient. Following treatment, the size of the tumor is measuring using MRI to determine if the tumor size has been reduced.

### Example 6: Combination Therapy Using a Targeting Agent and a PD1 Antibody Therapy to Treat Cancer

An individual with cancer is identified. A size of a tumor in the individual is measured by MRI with gadolinium contrast dye. First, a compound comprising a CD206 targeting agent and a cytotoxic agent is provided to the individual. Next, a PD1 antibody therapy is provided to the patient. Following treatment, the size of the tumor is measuring using MRI to determine if the tumor size has been reduced.

### Example 7: Combination Therapy Using a Targeting Agent and Antibiotics to Treat Tuberculosis

An individual with tuberculosis is identified. First, a compound comprising a CD206 targeting agent and a cytotoxic agent is provided to the individual. Next, one or more antibiotics are provided to the patient. Following treatment, a chest X-ray is performed on the individual to determine if the degree of tuberculosis infection has been reduced or eliminated.

### Example 8: Targeting Tumor Associated Macrophages (TAMs)

A prototype drug compound was designed using smaller targeting moieties with the size constraint of 10 kDa or less, as larger moieties, e.g., antibodies, which are about 150 kDa in size, will not pass into the vascular mimicry channels. The features of the prototype design are: targeting moieties (T), a backbone (B), and a payload (P). The backbone connects to the payload via a linker that is stable in the blood but releases the payload only within the CD206-expressing macrophages. The drug compound's ability to recognize and bind to CD206 gives the molecule its selectivity. The backbone enhances serum half-life, while the linker prevents release of the payload except within the endosome of target cells.

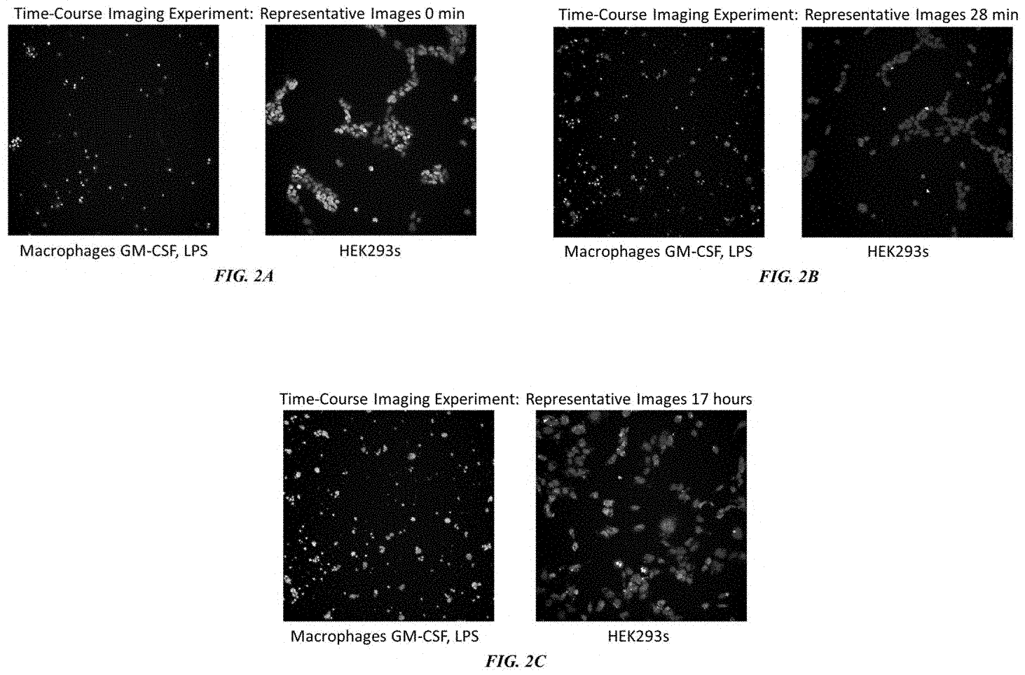

Time course endocytosis assays were performed utilizing attached and activated M2 macrophages to test whether the test drug compounds can be internalized by the macrophages. The cells were stained with the nuclear stain Hoechst, at 1 μg/mL final concentration and incubated for 30 minutes at 37° C. in a humidified incubator with 5% CO². Subsequently pHrodo™ Green Dextran was added to the cells and the cells were imaged at 0, 28, 49, 57, 67 minutes and 17 hours. The images shown in FIGS. 2A, 2B, and 2C represent the 0 min, 28 min, and 17 hour time points, respectively. The cell nuclei are depicted in blue and pHrodo™ Green Dextran in green. As pHrodo™ Green Dextran turns green only in the intracellular acidic environment, the images indicate that the dextran was internalized and was not simply bound to the exterior of the cells. The dextran continued to accumulate within the cell as a function of time (see the 17 hour time point in FIG. 2C).

Next, the test drug compounds were derivatized with fluorescein isothiocyanate (FITC) in order to detect the presence of compounds by confocal microscopy. FITC-conjugate drug compounds, TC-171-032 and TC-171-033, were tested for binding to and internalization by CD206⁺ macrophages and HEK293 cells (CD206⁻ control cells). Both test drug compounds have a dextran backbone (6 kDa molecular weight) with mannose as the targeting moiety, but different degrees of mannose moieties per molecule. In particular, TC-171-032 has 0.1 mol mannose/mol glucose and 0.001 mol FITC/mol glucose while TC-171-033 has 0.2 mol mannose/mol glucose and 0.01 mol FITC/mol glucose.

TABLE 3
Test drug compounds

| Molecule name | Test drug compound structure |
|---|---|
| 1. TC-171-032 | FITC_{0.001} Dextran_{6KD} Mannose_{0.10} |
| 2. TC-171-033 | FITC_{0.02} Dextran_{6KD} Mannose_{0.20} |
| 3. TC-171-082 | Lysine fixable version of TC-171-033 |

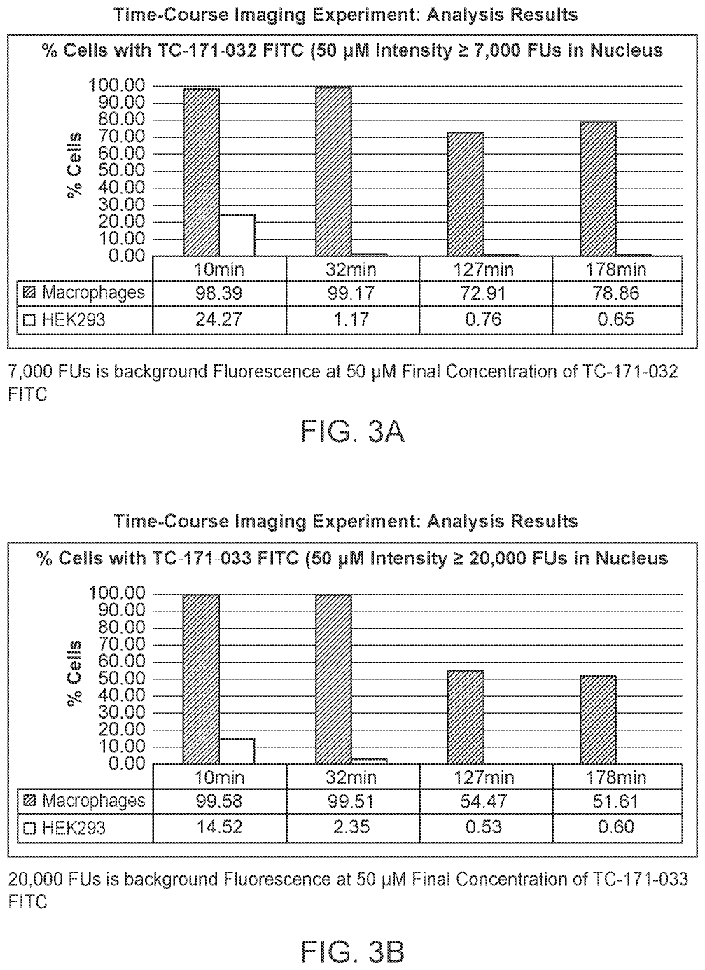

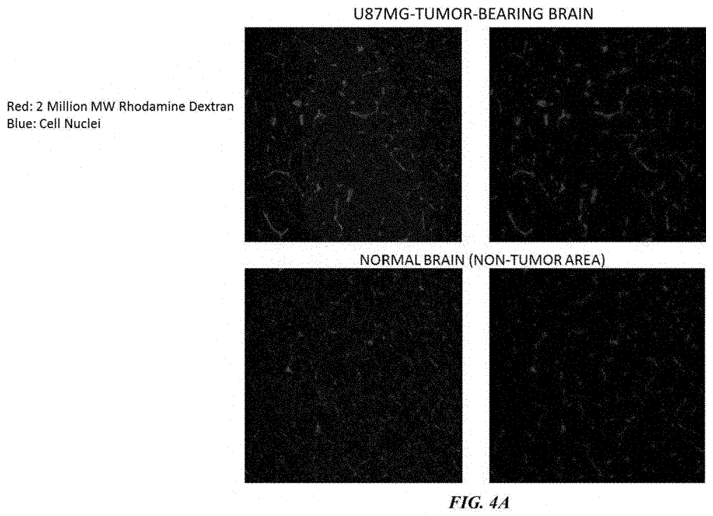

The graphs in FIGS. 3A and 3B represent experimental results obtained from an in-vitro macrophage internalization assay using TC-171-032 and TC-171-033, where the red bars represent macrophages and the blue bars represent the negative control cell line HEK293, which lacks CD206 expression. As shown in FIGS. 3A and 3B, macrophages internalized the fluorescently tagged test drug compounds TC-171-032 and TC-171-033 while HEK293 cells did not.

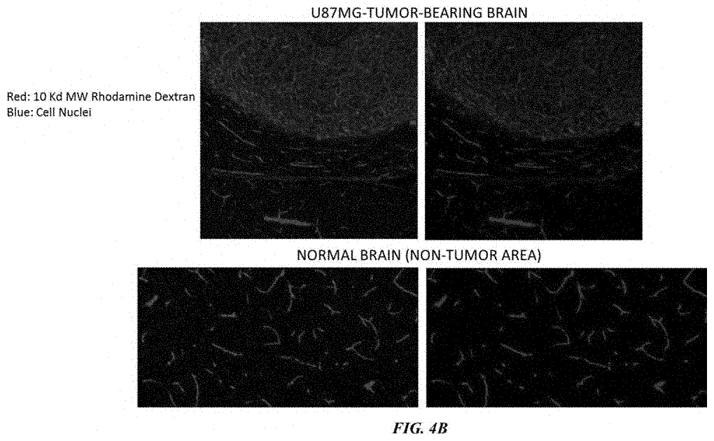

Next, to determine the appropriate size of the backbone molecule, rhodamine-dextrans of different sizes (molecular weights) were intravenously injected into experimental animals. In particular rhodamine-dextrans of 3,000, 10,000, 70,000, and two-million molecular weight (MW) were intravenously injected to U87MG tumor bearing mice and allowed to circulate systemically. Mice were sacrificed, and harvested brains were processed for confocal microscopy. As demonstrated by the images in FIGS. 4A and 4B, 10,000 MW rhodamine-dextran, but not the two-million MW rhodamine-dextran, entered the tumor parenchyma. The images also demonstrate that both 10,000 and two-million MW rhodamine-dextrans did not cross the normal blood-brain-barrier as these rhodamine-dextrans were not detected in normal brain parenchyma (FIGS. 4A and 4B, lower images). The results indicate that there is size selectivity for drug compound design as 3,000 MW (data not shown) and 10,000 MW rhodamine-dextrans crossed the blood-tumor-barrier while 70,000 MW (data not shown) and two-million MW rhodamine-dextrans did not. Moreover, 3,000 MW rhodamine-dextran localized with CD206⁺ tumor associated macrophages and the vascular mimicry network (FIGS. 1B and 1D).

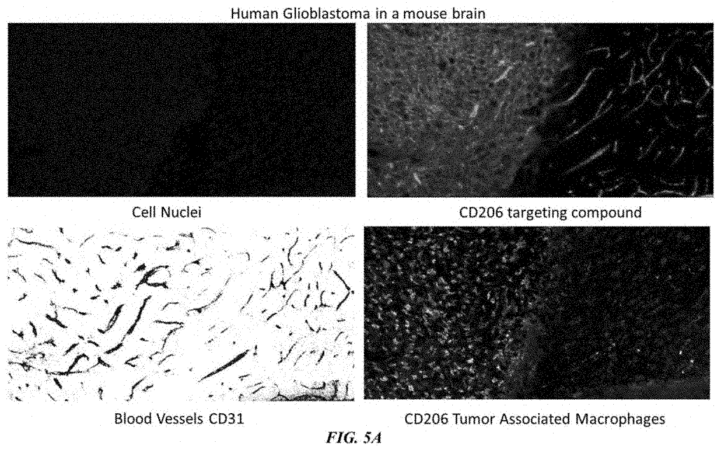

To determine if the test drug compounds target the tumor associated macrophages in the vascular mimicry network of both systemic and CNS tumors, test drug compounds were intravenously injected to mice bearing intracranial glioblastoma or subcutaneous melanoma. As demonstrated in FIGS. 5A and 5B, TC-171-082, a Lysine fixable version of TC-171-033, entered the tumor parenchyma in both glioblastoma and subcutaneous melanoma models. Importantly, in the brain, the drug compound only crossed the blood-tumor-barrier, but not the blood-brain-barrier. This drug compound, when conjugated with a cytotoxic agent in place of the FITC molecule, should be able to enter and kill tumor associated macrophages with reduced toxicity to normal brain tissue.

Experimental Procedures for U87MG Intracranial Injections in Athymic Nude Mouse:

Mice: Hsd (heterogenous outbred stock): Outbred Athymic Nude (J:NU) mice. Age: 4-6 weeks of age. Jackson Laboratories Cat #007850. Mice were housed under sterile conditions (sterilized cages and sterilized and irradiated food).

Cell line (U87-MG cells): Purchased from American Type Culture Collection (ATCC), Cat #HTB-14, Homo Sapiens, tissue=Brain, Disease=glioblastoma. Cells were grown in 10% Fetal Bovine Serum supplemented Eagle's Minimum Essential Medium (EMEM)+1× Penicillin/Streptomycin.

Preparation of cultured U87MG cells for intracranial cell injections: Cells were thawed, washed 1× incomplete media, and seeded in a sterile, canted neck, 75 cm² tissue culture flask. Cells were allowed to grow for 3-4 days and split 1:5 upon reaching confluency. Cells were harvested from their respective tissue culture flask at approximately 70% confluency with 3.0 ml of Tryp LE Express per flask for approximately 3-4 minutes at 37° C. Trypsin activity is stopped by adding 8 mls of complete media to each 75 cm² flask. Detached cells were collected with a sterile 10 ml stripette and cells were centrifuged for 4 minutes at 4° C. at 1,100 RPMs. Supernatant was then aspirated off and cells were washed 2× with sterile 1×PBS (containing cations). Cells were resuspended in 1×PBS and 500,000 cells per brain were injected intracranially in a volume of 5 μl using a Hamilton syringe.

Surgical Procedure:

1. The surface area of a ventilated Animal Transfer Station (ATS) was used as the surgical area. Prior to placing the KOPF stereotaxic apparatus and surgical instruments on the ATS, its surface was prepared by spraying all surfaces with a disinfectant and 70% Ethanol.
2. Prior to surgery, Mice bellies were swabbed with ethanol and mice were anesthetized with a 40 μl intraperitoneal injection of a Ketamine-Xylazine mixture in sterile saline.
3. Once a mouse is anesthetized, its scalp was prepared by swabbing it with a sterile alcohol prep pad (70% Isopropyl Alcohol). Eye ointment was applied to both eyes in order to maintain adequate moisture during the procedure. Using a sterile scalpel, a sagittal incision was performed over the head approximately 1 cm long. The exposed skull surface was then cleaned and dried using a sterile cotton swab applicator. After drying the cranial bones, the bregma became visible.
4. For intracerebral tumor establishment, a sterile 25-gauge sharp needle was used to puncture the skull to create a small hole in the cranium for the subsequent injection of tumor cells. Cells were injected into the brain using a 5 μl Hamilton Syringe at the following coordinates starting 3 mm right of the bregma, 1 mm anterior of the coronal suture and 3 mm deep from the surface of the cerebral cortex. Yet, the needle was brought down 3.5 mm from the surface to minimize the reflux of cells during the injection and to create a small pocket so that most of the injected cells stay 3 mm from the brain surface.
5. Prior to drawing cells into the syringe, the cell suspension was pipetted up and down 3-5 times using a 200 μl pipet and pipet tip. The syringe was slowly loaded with 5 μl of cell suspension in order to avoid creating air bubbles. The syringe was then placed perpendicular to the skull, over cranial hole previously created, lowered and the cell suspension was slowly injected at an approximate rate of 1 μl to 1.5 μl per minute. The needle was kept in place for another minute and slowly withdrawn in order to reduce reflux of the injected tumor cells.
6. The skull was cleaned and dried using a sterile dry cotton swab. Using sterile forceps, the scalp was drawn together over the skull and tissue glue was added to the incision. The scalp was then cleaned, and a triple antibiotic ointment was applied over the incision.
7. Mice were monitored post-operatively until they woke up from the anesthesia and normal activity was recovered.
8. Mice were housed for the duration of the study.
9. Once the study was completed, mice were euthanized, and brains were excised, washed 1× in sterile PBS, and processed for immunohistochemistry, flow cytometry, or both.
For flow cytometry, brain tissue was enzymatically digested; cells (brain and tumor) were isolated; and the live cell suspension was stained with fluorescein-conjugated primary antibodies for fluorescence-activated cell sorting analysis (FACS).
For immunohistochemical analysis, the brains were fixed overnight with 4% PFA/PBS at 4° C.
   10. Next day, brains were placed in a 15% Sucrose solution overnight at 4° C.
   11. Next day, brains were then transferred to a 35% Sucrose solution overnight at 4° C.
   12. Brains were then frozen in OCT and sectioned for Immunohistochemical analysis.

Immunohistochemistry:

60 μm-thick brain tissue sections were prepared with a Leica Cryostat, model CM1850. On average, 2-3 cryostat-tissue-sections were collected per glass slide and stored at −80° C. For immunohistochemistry analysis, slides were warmed, in darkness, on a dry 37° C. bath for approximately 10-15 minutes. Each slide was rinsed 1× with 750 μl/slide of 1×PBS with cations for 5 minutes at room temperature (RT). 1×PBS was gently removed and slides were blocked with 10% Normal Donkey Serum in 1×PBS (NDS/PBS) for 1 hour at RT. Blocking step was performed in darkness. After the blocking step, slides were rinsed with 500 μl/slide of 1×PBS (containing cations).

Approximately 700 μl/slide of primary antibody solution was added and incubated overnight at 4° C. in darkness. Antibody solutions were used in 2% NDS/PBS (with cations) at the following antibody dilutions:

Rat anti-mouse CD31 used at 1:25
Rabbit anti-mouse IBA-1 used at 1:100
Goat anti-mouse CD206 used at 1:50

All antibodies, with the exception of CD31 which was kept at 4° C., were from new aliquots from the −30° C. and were thawed on ice.

Next morning, slides were washed 3× with approximately 750 μl/slide/wash of 1×PBS with cations. Each wash lasted for 15 minutes at RT. Invitrogen's fluorescein-conjugated secondary antibodies were utilized at a 1:400 dilution in 2% NDS/PBS. These antibodies were donkey-anti-mouse, donkey-anti-rat, donkey-anti-rabbit, and donkey-anti-goat. Incubation was conducted overnight at 4° C.

Next day, sections were washed 3× with PBS and nuclei were stained with Hoechst 33342 for 15-20 minutes at RT in darkness. Sections were washed 3× with PBS and mounted on poly-L-Lysine-coated frosted slides with a drop of slowfade reagent. Appropriate no-secondary controls were performed in all experiments.

Confocal Microscopy:

Subcutaneous tumors sections were analyzed by Immunohistochemistry using confocal microscopy. All images were gathered with a confocal laser-scanning microscope (LSM 700 or 710, Carl Zeiss) utilizing a Plan-Apochromat 20×/0.8, Plan-Apochromat 63×/1.4 Oil DIC, C-Apochromat 40×/1.2W Korr UV-VIS objective lens (Carl Zeiss) and processed with the ZEN 2010 software (Carl Zeiss). Scanning was performed in sequential laser emission mode to avoid scanning at other wavelengths. Three-dimensional reconstructions were generated using ZEN 2010 and Imaris software (BitPlane, South Windsor, Conn.).

FACS/Flow Cytometry:

1. Resuspend cells at 8e6/ml in 1× D-PBS.
2. Add 1 μl/ml of fixable dead viability dye for 20 mins at RT.
3. Add FBS to achieve a 10% FBS-cells suspension with 1 μg/1e6 of mouse IgG (Jacksons Immunoresearch Cat #015-000-003) for 5 mins at RT. (Note: this step stops the reaction. In cells with compromised membranes, the dye reacts with free amines both in the cell interior and on the cell surface, yielding intense fluorescent staining. In viable cells, the dye's reactivity is restricted to the cell-surface amines, resulting in less intense fluorescence. The difference in intensity is typically greater than 50-fold between live and dead cells, allowing for easy discrimination.)
4. Add titration of antibody to the respective tube (antibody is directly conjugated with a different color than the viability dye).
5. Aliquot cell suspension (25 μl) to each respective tube and incubate on ice or at 4° C. for 10 mins.
6. Wash 3× with FACS Buffer (2% FBS/PBS/0.02% NaN3). Spin down the cells for 30 seconds at 3,000 rpm.
7. Remove soup with aspirator leaving 50 μl of FACS buffer/tube/wash.
8. Add 100 μl/tube of 1% PFA for 20 mins at RT. Samples can be run immediately but if you need to wait more than 2 days to run them, go to steps 10 & 11.
9. Wash 1× with D-PBS and spin down cells for 1 min at 3,000 rpm.
10. Remove soup with aspirator leaving 50 μl of FACS buffer/tube.
11. Add 100 μl of FACS buffer to each of the tubes.
12. Run samples on flow cytometry within 2-3 days.

Antibody titration instruction:

If the vendor suggests using an antibody at 5 μl/test, try the following amount of antibody for titration:

1. 5 μl of antibody
2. 1 μl of antibody
3. A 1:5 dilution of 1 μl of antibody in 1×D-PBS (discard 4 μl and add 1 μl to a Corning 1.2 ml polypropylene tube, 96 tubes/rack, Cat #: 4411).

Experimental Procedures for A-375 Melanoma Subcutaneous Tumor Model in Athymic Mice:

Mice: Hsd (heterogenous outbred stock): Outbred Athymic Nude (J:NU) mice. Age: 4-6 weeks of age. Jackson Laboratories Cat #007850.

Mice were housed under sterile conditions (sterilized cages with sterilized and irradiated food.

Cell line: A-375 Cells were purchased from American Type Culture Collection (ATCC), Cat #CRL-1619, Homo Sapiens, Human, tissue=Skin epithelial cells, Disease=malignant melanoma. Cells were grown in 10% Fetal Bovine Serum supplemented Dulbecco's Modified Eagle's Medium (DMEM)+1× Penicillin/Streptomycin.

Preparation of cultured A-375 cells for subcutaneous cell injections:

A-375 Human melanoma cells were thawed, washed 1× in complete media and seeded in a Corning sterile 75 cm2 tissue culture flask, canted neck. Cells were cultivated in complete media, composed of DMEM supplemented with 10% Fetal Bovine Serum (FBS) and 1× Penicillin/Streptomycin. For the first 72 hours post plating, cells grew slowly. However, cells began to proliferate quickly and looked healthy under the microscope after the initial 72 hours in culture. Cells were allowed to grow for 3-4 days and split 1:5, or 1:10, depending on the confluency and day of the week. Yet, for the most part, cells were split every 48-72 hours or as soon as they reached 85-90% confluency.

Cell splitting: Media was removed and pipetted into a 50 ml sterile centrifuge tube, and attached cells were rinsed with 3-4 mls of media per T75 and 3.5-4.0 mls/flask of Tryp-LE were added. Cells were incubated in Tryp LE Express for approximately 4 minutes at 37° C. Trypsin activity was then stopped by adding 8 mls of complete media per flask. The cell suspension was collected and centrifuged at 1,400 rpm for 5 minutes at RT. Supernatant was aspirated, and cells were resuspended and plated in a new T75s at either a 1:5 or 1:10 dilution in 10-12 mls of complete media.

Cell harvesting and subcutaneous injections: Cells were finally harvested and finally resuspended at 100×10⁶ cells/ml in DMEM media, antibiotic and serum free. Cell suspension was gently pipetted several times prior to injections and 50 μl of this cell suspension containing 5×10⁶ cells were subcutaneously injected into the right flanks of athymic mice using a Becton Dickinson ½ cc Insulin syringe with a 28G1/2 needle.

Mice were housed for the duration of the study (5 and 12 days). Following the conclusion of the study, A-375 subcutaneous tumors were harvested as follows:

Tumor bearing mice were intravenously injected by tail vein injection with 250 μl/mouse of Invitrogen's 10,000 MW dextran, Tetramethylrhodamine, Lysine fixable and/or TC Scientific's compound TC-171-082. The dextran and/or TC-171-082 solutions were allowed to circulate systemically in mice for either 2 or 3 minutes at RT, depending on the study, before mice were euthanized with Isoflurane followed by cervical dislocation.

Note: 25 mg of dextrans were dissolved in 1.0 ml of sterile saline. Syringes were loaded with 250 μl of a 25 mg/ml, 10,000 MW-rhodamine-dextran solution, and briefly stored at RT while wrapped in aluminum foil until their use. 250 μl of this dextran solution was intravenously administered by Tail vein injection to each mouse conforming this experimental group. Therefore, each mouse was injected with 6.25 mg of 10,000 MW rhodamine-dextran. The group of mice injected with TC-171-082 also received a 250 μl intravenous injection of a 27.5 mg/ml of compound in saline.

Tumors were excised and briefly rinsed with chilled 1×PBS on ice. They were then fixed with 4% PFA on ice for 2 hours and transferred to a 1% PFA/PBS solution overnight at 4° C. Next morning, 1% PFA/PBS solution was removed, tumors were rinsed with 4 mls of 1×PBS and placed overnight on a 15% Sucrose solution at 4° C. Next morning, tumors were transferred to a 30% sucrose solution and stored overnight at 4° C.

Immunohistochemistry:

60 μm-thick subcutaneous tumor sections were prepared with a Leica Cryostat, model CM1850. On average, 2-3 cryostat-tissue-sections were collected per glass slide and stored at −80° C. For immunohistochemistry analysis, slides were warmed, in darkness, on a dry 37° C. bath for approximately 10-15 minutes. Each slide is rinsed 1× with 750 μl/slide of 1×PBS with cations for 5 minutes at RT. 1×PBS was gently removed and slides were blocked with 10% Normal Donkey Serum (NDS) in 1×PBS with cations (NDS/PBS) for I hour at RT. Blocking step was performed in darkness. Slides were rinsed with 500 μl/each of 1×PBS containing cations.

Approximately 700 μl/slide of primary antibody solution was added and incubated overnight at 4° C. in darkness. Antibody solutions were used in 2% NDS/PBS (with cations) at the following antibody dilutions:

Rat anti-mouse CD31 (BD Pharmingen, cat #550274) used at 1:25.
Rabbit anti-mouse IBA-1 (Novusbio cat #NBP2-19019) used at 1:100.
Goat anti-mouse CD206 (R&D Systems, cat #AF2535) used at 1:50.
Rat anti-mouse CD11b (eBioscience, cat #14-0112) used at 1:100.
Rat anti-mouse F4/80 (eBioscience, cat #14-4801) used at 1:100.

All antibodies, with the exception of CD31 which was kept at 4° C., were from new aliquots from the −30° C. and were thawed on ice. Next morning, slides were washed 3× with approximately 750 μl/slide/wash of 1×PBS with cations. Each wash lasted for 15 minutes at RT.

Invitrogen's fluorescein-conjugated secondary antibodies were utilized at a 1:400 dilution in 2% NDS/PBS. These antibodies were donkey-anti-mouse, donkey-anti-rat, donkey-anti-rabbit, and donkey-anti-goat. Incubation was conducted overnight at 4° C.

Next day, sections were washed 3× with PBS and nuclei were stained with Hoechst 33342 for 15-20 minutes at RT in darkness. Sections were washed 3× with PBS and mounted on poly-L-Lysine-coated frosted slides with a drop of slowfade reagent. Appropriate no-secondary controls were performed in all experiments.

Confocal Microscopy:

Subcutaneous tumors sections were analyzed by Immunohistochemistry using confocal microscopy. All images were gathered with a confocal laser-scanning microscope (LSM 700 or 710, Carl Zeiss) utilizing a Plan-Apochromat 20×/0.8, Plan-Apochromat 63×/1.4 Oil DIC, C-Apochromat 40×/1.2W Korr UV-VIS objective lens (Carl Zeiss) and processed with the ZEN 2010 software (Carl Zeiss). Scanning was performed in sequential laser emission mode to avoid scanning at other wavelengths. Three-dimensional reconstructions were generated using ZEN 2010 and Imaris software (BitPlane, South Windsor, Conn.).

Procedure for Polarization and Activation of Macrophages

Cells: Human Peripheral Human Mononuclear cells (PBMC) (BIOIVT, BRH1322475)

Day 0: Start Macrophage Differentiation

1. Plate PBMC in Monocyte Attachment Medium (5 mL Medium per T-25 flask), using a seeding density of 1 million/cm2 for Mononuclear Cells with a monocyte content of >25% and 1.5 million/cm2 for a monocyte content of <25%. Use T-25 Nunc cell culture flasks with Nunclon™ surface. Incubate for 1.5 hours at 37° C. in a humidified incubator with 5% CO² without any further manipulation.
2. Prepare the complete Macrophage Generation Medium DXF by adding the entire content of the thawed SupplementMix aseptically to the Basal Medium. Swirl gently to obtain a homogeneous mixture. Immediately before use of the media, aseptically transfer the appropriate amount of Cytokine Mix M1 (100× stock) to the needed corresponding volume. Swirl gently until a homogeneous mixture is formed. The shelf life of the complete medium is 6 weeks at 4-8° C., and of complete medium with cytokines is 2 weeks.
3. Vigorously swirl the tissue culture flask to loosen non-adherent cells and aspirate them. Wash the adherent cells (i.e. monocytes) three times with warm Monocyte Attachment Medium by swirling the vessel and aspirating the supernatant.
4. Start the macrophage differentiation, by adding an appropriate amount of complete M1-Macrophage Generation Medium DXF to the cells (i.e. 7 mL per T-25 flask) and incubate for 4 days at 37° C. in a humidified incubator with 5% CO² without medium change. The monocytes differentiate to M1-like polarized macrophages under these conditions.

Day 4: Continue Macrophage Differentiation

Add another 50% to 75% by volume of fresh complete M1-Macrophage Generation Medium DXF to the cells. Incubate immature macrophages for another three days at 37° C. in a humidified incubator with 5% CO². Do not remove any of the used medium from the cells, just add the fresh medium.

Day 7: Activate Macrophages to a Specific Lineage

For specific M1 macrophage activation supplement the whole volume of the culture with LPS at a final concentration of 100 ng/mL. Do not perform a medium change, just add the activation factors.

Day 9: Medium Change

Aspirate the medium including suspension cells and collect it in a centrifugation tube. Immediately, pipet fresh complete Macrophage Generation Medium DXF supplemented with Cytokine Mix M1 (1×) and LPS (100 ng/mL). Centrifuge the cells in the tube for 15 min at 350×g at RT. Discard the supernatant and carefully resuspend the cells in a small amount of fresh medium. Combine the resuspended cells in the tube with the adherent cells in the flask. Incubate for at least one day at 37° C. in a humidified incubator with 5% CO². At this stage adherent and non-adherent cells may be observed.

Day 10:

According to PromoCell the macrophages may be used on Day 10. However, we have noticed that maintaining the cell in culture for one week, by performing a media change every 3 days, results in healthier and more macrophages for the end-assay. Macrophages can be maintained up to 2½ weeks in culture. Healthy cells will appear adherent with a prominent nucleus, flat outspread cytoplasm and multiple pseudopodia.

Day 10⁺: Harvesting of Macrophages for Cell-Based Assay

Aspirate and discard the medium from the flask. Wash the adherent macrophages with endotoxin-free PBS w/o Ca⁺⁺/Mg⁺⁺. Immediately add an appropriate amount of cold Macrophage Detachment Solution DXF to the cells; i.e. 8 mL per T-25 flask. Seal the flask and incubate cells for 1 hour at 4° C.; if necessary incubate at RT for another 20 min. Firmly tap the flask to facilitate cell detachment. Make sure most of the cells have already detached or are loosely adherent. Only then use a cell scrapper to dislodge the remaining macrophages. Collect the harvested macrophages in centrifugation tubes and dilute 1:1 with PBS/2 mM EDTA/0.1% HSA. Centrifuge cells for 15 min at 350×g at RT. Apply one wash with PBS/2 mM EDTA/0.1% HSA to the cells and count them. The macrophages are now ready to be used for your cell-based assay.

Note: The detachment process can affect the number of viable macrophages that can be harvested. Make sure to allow enough time for the Macrophage Detachment Solution to facilitate cell detachment before dislodging with a cell scrapper.

Procedure for Cell-Based Endocytosis Assay with Human Macrophages

Cells:

### Human M1-Macrophages (Generated Based on ‘Polarization and Activation of Macrophages’ SOP)

### Human Embryonic Kidney Cells (HEK293) (ATCC, ATCC® CRL-1573™)

### Experimental Procedure:

Day 1

1. Dilute the Fibronectin Solution (stock 1 mg/mL) to 10 μg/mL final concentration in PBS w/o Ca⁺⁺/Mg⁺⁺. Add 20 μL of diluted Fibronectin Solution per 384-well. Place plate(s) on a level surface at RT for 60 min. Aspirate the excess Fibronectin Solution. Use fibronectin-coated plate(s) immediately or let air-dry under a laminar flow bench and store at 4° C. for up to 2 weeks.
2. Dilute harvested macrophages and HEK293 cells to a density of 160,000 cells/mL (4,000 cells/well in 254), in their respective growth media. Assure cytokines and LPS are added to complete M1-Macrophage Generation Medium DXF. Add 25 μL of cells to desired wells of the Perkin Elmer LLC CellCarrier-384 Ultra Microplate(s) and allow cells to adhere overnight.

Day 2

1. Next morning add compounds (diluted in DMSO) to desired wells, with the Echo® 555 Liquid Handler, using a ten point three fold dilution series and a top concentration of 50 μM. Assure specific wells are treated only with DMSO. Immediately after, add Hoechst to all plate wells at a concentration of 1 μg/mL in a final volume of 50 μL.
2. Incubate cells with compounds and Hoechst for 10 minutes at 37° C. in a humidified incubator with 5% CO².
3. Image plate wells with the Opera Phenix™ High Content Screening System using confocal imaging with a 20× water objective, 9 fields per well, and the Hoechst and Alexa 488 filters. Image wells 10 min, 30 min, 1 hour, 2 hours, and 3 hours after Hoechst addition. 4. Analyze images with the Columbus Image Data Storage and Analysis System to generate:

quantitative measures of compound fluorescent intensity in the nucleus and cytoplasm of macrophages and HEK293 cells, and to

identify number of cells with compound fluorescent intensity above background for macrophages and HEK293 cells.

5. Use Microsoft Excel to compare quantitative data for macrophages and HEK293 cells in graphic format.

### Example 9: CD163-Expressing Tumor Associated Macrophages in Human Malignant and Benign Meningioma

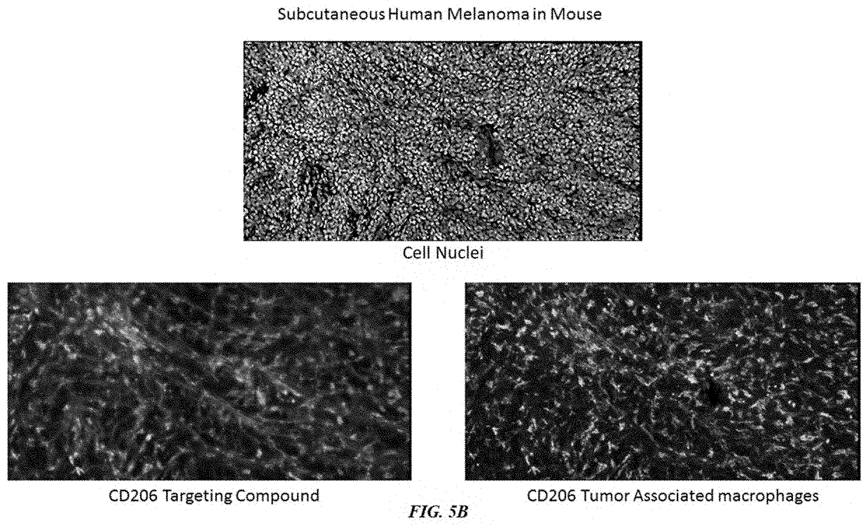

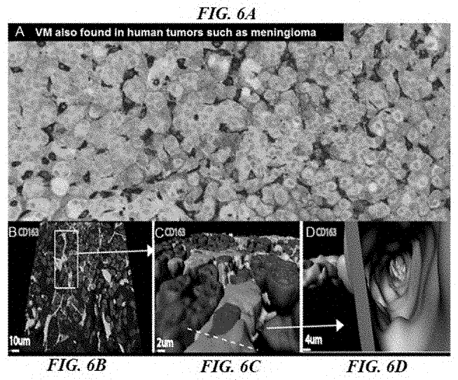

Tumor tissues from a 56-year-old male with malignant meningioma (FIG. 6A) and a 44-year-old female with a benign (grade I) meningioma (FIGS. 6B-6D) were obtained. Tissues were sectioned at 6 μm thickness and stained with an anti-CD163 antibody. Both positive and negative controls were included and valid. FIG. 6A demonstrates that a CD163+ interconnected macrophage network is present in malignant meningioma (Hematoxylin stain in light grey and CD163 immunoperoxidase in dark grey). FIG. 6B shows an Imaris three-dimensional (3D) rendition of anti-CD163 immunoperoxidase-stained tissue from the 44-year-old female with a benign meningioma. Light gray colored structures depict CD163-expressing tumor associated macrophages and dark gray colored structures depict cell nuclei. FIG. 6C shows an enlarged view of the square in FIG. 6B showing a vascular mimicry channel formed by CD163-expressing tumor associated macrophages. FIG. 6D shows a cross-sectional view of the lumen associated with a CD163-expressing-macrophage-lined vascular mimicry tubular structure.

### Example 10: Formation of Perfuse Vascular Mimicry Tubular Network by Macrophages in Low Oxygen Environments

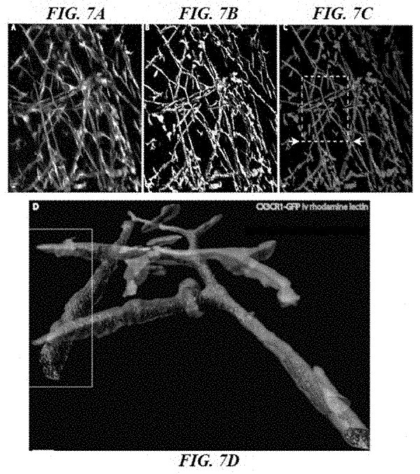

Mice were subcutaneously injected with a Matrigel solution which solidifies at body temperature and forms a subcutaneous plug. The subcutaneous avascular plug has a low oxygen environment within it and macrophages, which migrate into the plug after polymerization, form a three-dimensional (3D) tubular network. A few days after Matrigel solution injection, a fluorescent dextran was intravenously injected into the live mouse and the dextran was allowed to circulate systemically for a few minutes. FIG. 7A shows a confocal z-stack image of a macrophage 3D tubular network in experimental mice, indicating that in low oxygen environments, such as in subcutaneous Matrigel and various forms of tumors, macrophages can form a functional, or perfused, 3D network that serves as a form of vascular mimicry. FIG. 7B shows an Imaris 3D representation of the image in FIG. 7A. FIG. 7C shows the image in FIG. 7B made transparent to show that the intravenously-injected-fluorescent-dextran can circulate within the vascular mimicry tubes. FIG. 7D shows an enlarged view of the doted-square in FIG. 7C where the image in FIG. 7C was rotated along the horizontal axis to show that the macrophage vascular mimicry channels are perfused with the intravenously injected fluorescent dextran.

### Example 11: Formation of a Granuloma-Like Structure by Macrophages

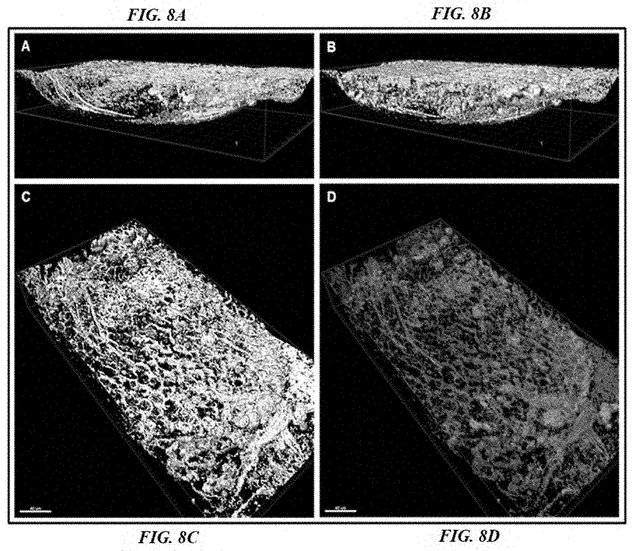

Imaging studies of mouse uveal melanoma (B16F10, murine melanoma from a C57BL/6J mouse) showed that a macrophage network forms a granuloma-like structure (FIG. 8A) encasing uveal melanoma (FIG. 8B). Intravenously injecting 3,000 MW rhodamine-dextran resulted in a macrophage network perfused with the dextran (FIG. 8C). The image in FIG. 8C made transparent with Imaris 3D imaging software is shown in FIG. 8D. A patient with macrophages with granulomatous structure encasing the tumor can be treated with a drug that targets the macrophages that are encasing the tumor and covering the tumor-associated antigens with “self” molecules. The macrophages encasing the tumor can be stripped off by targeted killing and the tumor antigens can be more accessible for treatment with various immunotherapeutics such as Car-T therapy/anti-PD1 or anti-PDL1 antibodies (Examples 5 and 6).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1.-150. (canceled)

151. A composition comprising: (i) a targeting moiety capable of targeting a tumor associated macrophage and (ii) a cytotoxic agent, wherein each of (i) and (ii) is attached to a dextran backbone, and wherein the composition is less than 20 kDa in size.

152. The composition of claim 151, wherein the cytotoxic agent is attached to the dextran backbone via a linker.

153. The composition of claim 152, wherein the linker comprises a disulfide bond.

154. The composition of claim 151, wherein the targeting moiety comprises a CD206 ligand.

155. The composition of claim 154, wherein the CD206 ligand comprises mannose, fucose, sulfated N-acetylgalactosamine, N-acetylglucosamine, luteinizing hormone, thyroid stimulating hormone, a chondroitin sulfate, or any combination thereof.

156. The composition of claim 154, wherein the CD206 ligand comprises mannose.

157. The composition of claim 151, wherein the targeting moiety comprises a CD204 ligand.

158. The composition of claim 157, wherein the CD204 ligand comprises lipid A, oxidized low-density lipoprotein (LDL), acetylated LDL, malondialdehyde modified LDL, maleylated LDL, lysophasphatidylcholine, phophatidic acid, cholesterol, Apo A-I, Apo E, glycated type IV collagen, modified collagen type I, III and IV, biglycan, decorin, albumin, advanced glycation end product bovine serum albumin, β-amyloid fibrils, calreticulin, gp96, an HSP70 protein, a lipopolysaccharide, lymphotoxin-alpha, CpG DNA, calciprotein particles, a Neisseria meningitides surface protein, C reactive protein, hepatitis C virus NS3 protein, Tamm-Horsfall protein, or any combination thereof.

159. The composition of claim 157, wherein the CD204 ligand comprises glycated type IV collagen, modified collagen type I, modified collagen type III, modified collagen type IV, or any combination thereof.

160. The composition of claim 151, wherein the cytotoxic agent comprises a chemotherapeutic agent, an anti-tubulin agent, a DNA modifying agent, a small interfering ribonucleic acid, or any combination thereof.

161. The composition of claim 151, wherein the cytotoxic agent is selected from the group consisting of an auristatin, a dolastatin, auristatin E, Monomethyl auristatin E (MMAE), Monomethyl auristatin F (MMAF), dimethylvaline-valine-dolaisoleuine-dolaproine-phenylalanine-p-phenylenediamine (AFP), 5-benzoylvaleric acid-auristatin E ester (AEVB), (AEB), Ansamitocin, Mertansine/emtansine (DM1), ravtansine/soravtansine (DM4), Duocamycin, Calicheamicin, and pyrrolobenzodiazepine.

162. The composition of claim 151, wherein the cytotoxic agent comprises Monomethyl auristatin E (MMAE).

163. A method of treating cancer in a subject comprising administering a therapeutically effective amount of the composition of claim 151 to the subject.

164. A method of disrupting a macrophage network comprising one or more macrophages, the method comprising contacting the macrophage network with the composition of claim 151.

165. A method of delivering an agent to a tumor comprising administering a composition comprising the agent and a targeting moiety to a subject, wherein the composition is capable of entering and perfusing through a macrophage network.

166. The method of claim 165, wherein the agent is an imaging agent.

167. The method of claim 166, wherein the imaging agent is dextran-tetramethylrhodamine.

168. The method of claim 165, wherein the composition comprises a targeting moiety capable of targeting a tumor associated macrophage.

169. The method of claim 165, wherein the composition is less than about 20 kDa.

170. The method of claim 165, wherein administering comprises intravenous injection.
